(19) United States
(12) Patent Application Publication
(10) Pub. No.: US 2019/0159912 A1
(43) Pub. Date: May 30, 2019

(54) METHOD AND DEVICE FOR TREATING A TARGET SITE IN A VASCULAR BODY CHANNEL

(71) Applicant: Quadra Endovascular, Inc., Grand Junction, CO (US)

(72) Inventor: Richard E. Fulton, III, Grand Junction, CO (US)

(21) Appl. No.: 16/018,442

(22) Filed: Jun. 26, 2018

Related U.S. Application Data
(63) Continuation of application No. 14/734,294, filed on Jun. 9, 2015, now abandoned.
Application No. 14/734,294 is a continuation-in-part of application No. 14/231,014, filed on Mar. 31, 2014, now Pat. No. 9,114,031.
Application No. 14/231,014 is a continuation of application No. 12/813,339, filed on Jun. 10, 2010, now Pat. No. 8,740,961.
(60) Provisional application No. 62/009,400, filed on Jun. 9, 2014.
(60) Provisional application No. 61/277,154, filed on Sep. 21, 2009.
(60) Provisional application No. 61/274,165, filed on Aug. 13, 2009.

(51) Int. Cl. A61F 2/82; A61F 2/01; A61F 2/958; A61B 17/22

(52) U.S. Cl. CPC A61F 2/82; A61F 2/013; A61M 2025/105; A61B 17/22; A61F 2/958

(57) ABSTRACT

A novel temporary vascular scaffold is described. The temporary vascular scaffold is placed over an existing balloon catheter assembly, such as a commercially available balloon catheter assembly, and attached thereto. The temporary vascular scaffold is placed over the balloon of the catheter assembly. The balloon and the temporary vascular scaffold are expanded against a target region in the vessel wall. The temporary vascular scaffold causes the balloon expansion to be more gradual and well distributed over the outer surface of the balloon, reducing the risk of trauma. The balloon is then collapsed, leaving the temporary vascular scaffold to provide support to the vessel wall. While the vessel wall is propped open, the target region is infused with a diagnostic or therapeutic agent to diagnose or treat the target region.

### CROSS-REFERENCE

This application is a continuation of U.S. application Ser. No. 14/734,294, filed on Jun. 9, 2015, which claims the benefit of U.S. Provisional Application No. 62/009,400, filed Jun. 9, 2014, which applications are incorporated herein by reference in their entirety, and is also a continuation-in-part application of and claims the benefit of U.S. patent application Ser. No. 14/231,014, filed Mar. 31, 2014, which application is a continuation of U.S. patent application Ser. No. 12/813,339, filed Jun. 10, 2010, which application claims the benefit of U.S. Provisional Patent Application Nos. 61/274,165, filed on Aug. 13, 2009, and 61/277,154, filed on Sep. 21, 2009, which applications are incorporated herein by reference in their entirety.

### BACKGROUND

Balloon angioplasty has been a popular method of treating vascular occlusions since 1976. With plain old balloon angioplasty (POBA), there exists a significant subset of patients who have immediate suboptimal results related to the trauma to the vessel including dissection of the vessel, incomplete plaque compression, poor lumen gain, and acute elastic recoil of the vessel, amongst others. Because of these suboptimal immediate results, other means to treat vascular stenosis were developed. Intravascular stents are widely utilized, addressing the acute problems of angioplasty and reducing the restenosis rates from 50-60% for POBA to 30-35% for these bare metal stents (BMS).

Because the restenosis rates of BMS are usually unacceptable, drug eluting stents (DES) are used to inhibit restenosis. These devices reduce the restenosis rate to around 20% and lower in the coronary circulation. However, DES are extremely expensive and can lead to thrombosis, which can prove fatal. In addition, DES are not particularly effective in the peripheral circulation. The expense of drug eluting stents dramatically increases the overall cost of healthcare in the U.S. Finally, not only are the stents are costly, but expensive and potentially harmful drugs are routinely used for at least a year after stent implantation.

It is apparent that restenosis is the Achilles heel of all vascular intervention, from angioplasty to stenting and even surgery. However, it is clear than drugs can prevent restenosis. The primary question is how best to deliver the drugs in the most cost effective manner available while producing good patient outcomes and preventing complications.

Because of the variable plaque morphology and composition, stresses provided by conventional POBA are unpredictable, and frequently high pressure balloon inflations are needed to successfully provide enough stress to crack the plaque. When the plaque does compress at high pressures, the balloon will very rapidly expand to its full dimension in a noticeable pop (tenths of a second), very rapidly expanding the vessel wall often rupturing the smooth muscle cells. Dissection frequently occurs, as does irreparable injury to the smooth muscle cells which do not have the chance to gradually stretch and deform to maintain their integrity.

Therefore, methods and devices that lower the pressure at which the plaque will fracture will produce a slower and more gradual stretching of the arterial wall. This slower stretching will diminish the degree of trauma to the vessel.

A wire or wires along the outside of an angioplasty balloon, sometimes called buddy wires, produces focal areas of stresses along the wires that were approximately 120 times that of a conventional balloon surface, and the stress patterns from the external wire extends into the plaque rather than being concentrated on the surface as with a conventional balloon. The stress patterns are less dependent on the morphology and composition of the plaque than with conventional balloons. In other words, the stresses are more predictable, concentrated, and required lower balloon pressures to compress the plaque. Clinical studies confirmed that when compared to conventional POBA catheters, the buddy wire technique compressed the plaque at lower balloon pressures, caused fewer dissections, had less elastic recoil, and had more lumen gain, as well as a trend toward lower restenosis rate.

More recently, cutting and scoring balloons have been introduced extending these concepts. One such balloon uses several razor type blades along the balloon margins. Scoring balloons utilize several 0.005 to 0.007 inch struts placed over a balloon. Both balloon types are commercially successful. They are typically used in treating complex lesions or in plaque modification. The scoring balloon has been shown to achieve 50% more lumen gain than POBA when utilized as predilatation before stent implantation. This procedure significantly reduces the number of dissections when compared to POBA. The scoring balloon also has been shown to not slip off of the lesion, which is a problem with POBA. The scoring balloon is also more effective in soft, fibrous, and calcified plaques than POBA and has been recommended as a strategy of plaque modification in treating complex lesions. The use of the scoring balloon has thus resulted in very low incidences of inadvertent or unplanned stenting, commonly referred to as bail out stenting.

Prolonged inflation times improve the immediate results of POBA with fewer dissections, fewer further interventions such as stenting, and less restenosis. On the other hand other studies did not show improvement in long term results with prolonged inflation times, possibly because their prolonged inflations were the result of treating dissections. Applicant is unaware of any studies that evaluate both plaque modification and prolonged inflation times.

While these mechanical strategies have resulted in measurable improvement in the acute complications of POBA, the most promising advancement in POBA has been the advent of drug eluting balloons (DEB's), which for convenience purposes will be discussed as being synonymous with a drug coated balloon (DCB). A DEB is a POBA balloon coated with an antiproliferative drug, such as paclitaxel. The drug is delivered during the rather short balloon inflation and is present in smooth muscle cells up to six days later. The drug from a DEB covers essentially 100% of the plaque/vessel wall vs. only 15-20% with drug eluting stents. Compared to DES in treating coronary in-stent restenosis, a DEB seems preferable. In the THUNDER trial (sponsored by University Hospital Tuebingen, Tuebingen, Germany, reported in The New England Journal of Medicine, volume 358:689-699, Feb. 14, 2008, Number 7), a DEB was compared to POBA in the peripheral vasculature. DEB was very effective, and at 2 years the target lesion revascularization rate was only 15% with the DEB vs. 59% with POBA. Most experts in the field expect the general usage results of DEB's in coronary circulation to be in the range of drug eluting stents, i.e., a restenosis rate of around 20% or so. This rate leaves considerable room for improvement.

Therefore, both mechanical and pharmacological strategies have shown advantages in treating vascular lesions with balloon angioplasty. The mechanical strategies effectively address the acute or immediate problems by causing less injury to the vessel and the pharmacological strategy of drug eluting balloons significantly diminish restenosis.

Moreover, recent experiments have demonstrated that infusion of paclitaxel, an antiproliferative drug, directly into the artery may be just as effective as drug eluting balloons or drug eluting stents. This is usually done by employing a catheter specifically designed for infusion of a drug over the site of the angioplasty or stent placement after the angioplasty and/or stent placement. This type of catheter usually has two balloons, one proximal and one distal. The drug or other agent is infused between the two in a closed system, drug infusion performed after the angioplasty, stent placement or other therapeutic procedure. This requires removal of the angioplasty balloon or stent delivery catheter, which is utilized prior to the drug delivery, and subsequent placement of a separate device to deliver the drug. This is problematic not only because of the cost of the extra device, but also platelets adhere over the fissures in the plaque and about the small areas of injury in the arterial wall while the exchange is taking place, preventing some of the drug from being delivered to the wall where it is needed. Additionally, by just infusing a drug into a space that has been previously dilated, there is very little pressure forcing the drug into the wall. Subsequent to the therapeutic procedure and the drug delivery steps, the drug is then released downstream.

In U.S. Pat. No. 5,059,178, Ya et al. describe a device with a downstream balloon catheter blocking element and an upstream suction catheter with a balloon blocking element for the removal of thrombus from a blood vessel. The device is utilized to dissolve the thrombus by injecting a dissolving agent into the space between the two balloons and then withdraw the dissolved thrombus from the body through upstream suction catheter. Any subsequent intervention or therapy (angioplasty, stent placement, and the like) are performed after the removal of the dissolved thrombus.

In U.S. Pat. No. 6,022,366, Zadno-Azizi et al. describe another double balloon device similar to one described by Ya above but is directed toward embolic containment. This device is actually a three catheter irrigation/aspiration system and also has an innermost downstream balloon blocking or occluding element and an outermost upstream balloon occlusion catheter with an intermediate catheter between the two. The irrigation/aspiration of debris and emboli occurs by use of the outer pathway between the upstream balloon occlusion catheter and the intermediate catheter, and by the use of the inner pathway between the intermediate catheter and the innermost downstream balloon blocking element. The use of three catheters tends to reduce the cross-sectional size of the pathway available for aspiration of material.

In U.S. Pat. No. 5,449,372, Schmalz et al. describe a temporary stent that can be used for support after dilatation of the lesion.

### SUMMARY

To address the problem of how best to deliver the drugs in the most cost effective manner available while producing good patient outcomes and preventing complications, the medical device industry has essentially focused on developing methods and devices that inhibit the vascular response to the injury (restenosis), as opposed to developing a device that causes less injury, and hence less restenosis. Aspects of the present disclosure are directed to a device and method that both 1) can cause less injury to the vasculature by the use of dilatation of a braid over a balloon causing less dissection and more even plaque disruption at lower pressures and 2) can introduce drug deep within the vessel wall; this latter act may be accomplished by using proximal and distal occluders, injecting an agent, such as an anti-proliferative drug, into the region between the occluders, and performing an intervention, such as balloon angioplasty, while the occluders and injected agent remain in place. Other aspects of the present disclosure include providing help to maintain pressure upon the vessel wall similar to prolonged balloon inflation by using a braided, stent like structure as a temporary or transient stent. Thus, less initial injury and less elastic recoil should result in less restenosis, and further delivering a drug can further reduce or prevent the restenosis.

It may be the immediate result of an intervention (the immediate lumen diameter and the immediate residual percent stenosis) that typically determines the late outcome after coronary or other vascular intervention. Devices of the present disclosure can be adapted to improve these two factors. An optimal outcome in percutaneous interventions may depend upon: 1) obtaining excellent acute angiographic results with less dissection and elastic recoil, 2) avoiding damage to the distal vascular bed (as with atherectomy), and/or 3) reducing smooth muscle cell proliferation with pharmacological intervention. The systems, devices, and methods of the present disclosure can address one or more of these three areas, for example, all three areas.

Embodiments of the present disclosure may be directed to a method of treating a target site within a vascular channel of the body using a catheter assembly, the catheter assembly comprising a proximal occluder and a distal occluder. The method may include the following steps. The proximal occluder may be positioned in a vascular channel-occluding state within the vascular channel at a first position proximal of a target site thereby occluding the vascular channel at the first position. The distal occluder may be positioned in a vascular channel-occluding state within the vascular channel at a second position distal of a target site thereby occluding the vascular channel at the second position and thereby defining a region between the distal and proximal occluders. An agent may be introduced (e.g., by injection) into the region. An intervention may be performed at the target site while the distal and proximal occluders are in their vascular channel-occluding states and the agent is in the region. The catheter assembly may then be removed from the vascular channel.

In some examples, the intervention performing step comprises expanding an expansion device, such as a balloon and/or a temporary stent structure covering the balloon, against an inner wall of the vascular channel. In some examples, the balloon is collapsed leaving the stent structure expanded against the inner wall for a period of time, and the collapsed balloon and the collapsed stent structure are removed from the vascular channel during the stent structure removing step.

An example of a balloon stent assembly may comprise a catheter assembly having a proximal portion and a distal portion. The catheter assembly may comprise first and second elongate members. A temporary stent may have proximal and distal ends; the proximal end may be secured to a first position along the first elongate member and the distal end may be secured to a second position along the second elongate member, the temporary stent may be placeable in a contracted state by movement of the first and second positions away from one another. The assembly may also include an inflatable balloon mounted to the distal portion of the catheter assembly at a location surrounded by the temporary stent. The balloon may be placeable in an inflated state, thereby placing the temporary stent in an expanded state, and in a collapsed state. The temporary stent can be free to remain in the expanded state when the balloon moves to the collapsed state.

By utilizing the balloon to expand the temporary stent, not only the pressure of the balloon can be brought to bear on the obstruction, but its actions can be enhanced by the overlying temporary stent structure. The wires of the temporary stent may provide areas of focal force on the plaque that may allow the plaque or obstruction to be dilated with less pressure creating a controlled expansion compared to the uncontrolled rupture and dissections frequently seen with POBA. There may be a more gradual stretching and more gradual deforming of the smooth muscle cells, and they may have an opportunity to accommodate this stretching and maintain their integrity rather than being irreparably injured as is frequently the case with POBA. Therefore the balloon can serve two distinct functions: 1) it can dilate the plaque or obstruction (and in a more consistent manner because of the overlying temporary stent structure), and 2) it can dilate the temporary stent more effectively, with more force, and with more lumen gain than could be achieved by dilating the temporary stent structure without the assistance of the balloon. Therefore together the balloon along with the temporary stent can be able to effectively dilate and then support the dilated vessel subsequent to the dilatation.

In some examples, the first elongate member comprises an outer, actuator sleeve and the second elongate member comprises an inner, balloon catheter shaft to which the balloon is mounted. In some examples, the temporary stent comprises a porous braided stent structure.

Treating advanced vascular disease is one of the largest health care expenses born by society. There are projected to be one million non-coronary angioplasties and 900,000 stand-alone coronary angioplasties in 2012. (Millennium Research Group, 2009. American Heart Association, Heart Disease and Stroke Statistics, 2009 Update at a Glance.) These simpler, less expensive interventional methods, such as POBA, are frequently not effective, necessitating the use of more complex and expensive alternatives, such as stenting and surgery, which cost billions of dollars each year.

The use of the systems, devices, and methods of the present disclosure can be expected to improve on the results of POBA and reduce or avoid the need for stenting and/or surgery, by causing less vascular injury initially, preventing elastic recoil that frequently demands stenting, and preventing restenosis by simultaneously administering a non-proliferative agent. A procedure conducted according to the present disclosure may be expected to cost only marginally more than POBA.

A rough calculation may show that the use of the systems, devices, and method of the present disclosure could result in large cost savings of over $1 billion per year as approximately 1.9 million peripheral angioplasties and stand-alone coronary angioplasties (not associated with stent implantation) will be performed in 2012. (Millennium Research Group, 2009. American Heart Association, Heart Disease and Stroke Statistics, 2009 Update at a Glance.) By replacing POBA with the procedures described herein in all cases, and diminishing the re-intervention rate from 40% of 1.9 million patients (760,000 patients) to 10% (190,000 patients), approximately 570,000 patients may be spared re-intervention. At a Medicare reimbursement cost of $5850/procedure, there may be savings of $3.33 billion/year. Currently, such restenotic lesions are usually treated with stents, surgery, or other more costly methods. On average, these added procedures add a cost of about $2,000 for each procedure. If the $2000 is added to each re-intervention in 80% of these cases, then the savings can be increased by $912 million (570,000 procedures X 80% X $2000=$912 MM), for a total possible savings of $4.24 billion per year. A market penetration of 25% may result in yearly cost savings of over $1 billion per year, not even considering the expected diminished incidence of costly “bail out” or unanticipated stenting when using the systems, devices, and methods of the present disclosure.

Aspects of the present disclosure provide methods of treating a target region within a bodily lumen. A stent-like tubular scaffold assembly may be advanced over an expandable element on a distal portion of a catheter. The stent-like tubular scaffold assembly may be removably or fixedly affixed to an elongate shaft of the catheter. The catheter and the stent-like tubular scaffold assembly may be positioned at or near the target region. The expandable element of the catheter may be expanded to press the expandable element and the stent-like tubular scaffold assembly against the target site. The expandable element may be collapsed while leaving the stent-like tubular scaffold assembly expanded against the target site. The expanded stent-like tubular scaffold assembly may maintain sufficient pressure against an inner wall of the bodily lumen at the target site to inhibit elastic recoil thereof. The stent-like tubular scaffold assembly may then be collapsed.

The stent-like tubular scaffold assembly may be affixed to the catheter shaft by affixing a proximal affixation member and/or a distal affixation member of the stent-like tubular scaffold to the elongate shaft. The proximal affixation member may comprise a ring-like proximal structure. In some embodiments, the proximal affixation member may be adjustable so that it may prevent any translation, partial translation, or free translation of the proximal affixation member relative to the elongate shaft. In some embodiments, the catheter shaft may comprise an inner elongate catheter shaft and an outer elongate catheter shaft which may be translated relative to one another. The proximal affixation member may be coupled to a distal portion of the outer elongate catheter. The distal affixation member may be coupled to a distal portion of the inner elongate catheter.

The distal affixation member may comprise a ring-like distal structure. The ring-like structures may be affixed to the elongate shaft with an interference fit. In some embodiments, the ring-like structure(s) may comprise an elastic band, and wherein the stent-like tubular scaffold assembly may be affixed to the elongate shaft by expanding the elastic band, advancing the elastic band over the elongate shaft, and leaving the elastic band without constraint so that the elastic band holds the stent-like tubular scaffold assembly in place against the shaft of the catheter. In some embodiments, the ring-like structures may comprise a ratchet mechanism, and the stent-like tubular scaffold assembly may be affixed to the elongate shaft by opening the ratchet mechanism, advancing the ratchet mechanism over the elongate shaft, and closing the ratchet mechanism so that the ratchet mechanism holds the stent-like tubular scaffold assembly in place against the shaft of the catheter. In some embodiments, the distal affixation member may be comprised of ridges or barb-like structures about the inner surface of the distal shaft of the stent-like tubular scaffold assembly or projections that may be associated with the attachment of the distal shaft to the scaffold and may extend through the wall of the distal shaft. The ridges or barb-like structures may create friction with the outer surface of the elongate shaft of the expandable element catheter and/or the ridges or barb-like structures may be shaped to penetrate the outer surface of the coaxially encompassed distal elongate shaft of the expandable element catheter which may prevent any translation of the inner and outer distal components. These ridges or barb-like structures may be angled, may be pointed, may be circumferential or non-circumferential, may be oriented in patterns including, but not limited to, a screw like pattern and may be oriented toward the center of the outer elongate shaft or to the proximal, distal, or radial aspect of the outer elongate shaft or a combination of the above. In some embodiments, the distal fixation mechanism may comprise other fixation mechanisms that preferentially affix the inner aspect of the distal outer shaft of the stent-like tubular scaffold assembly to the outer aspect of the inner positioned elongate member of the expandable element catheter.

Expanding the expandable element of the catheter typically concurrently expands the stent-like tubular scaffold assembly. The stent-like tubular scaffold assembly may distribute expansion forces of the expandable element to reduce focal points of expansion over an outer surface of the expandable element and to reduce undesired trauma to an inner wall of the bodily lumen at the target region as both the expandable element and the stent-like tubular scaffold assembly are expanded. The stent-like tubular scaffold assembly may limit the expansion of the expandable element or cause the expansion to be more gradual than without the stent-like tubular scaffold assembly, thereby more gradually stretching and deforming the inner wall of the bodily lumen at the target site to reduce a risk of trauma thereto. The stent-like tubular scaffold assembly may also compress the expandable element so that it may assume a smaller profile state upon deflation and collapse of the expandable element.

The stent-like tubular scaffold assembly may be collapsed a period of time after the expandable element of the catheter is collapsed, leaving the stent-like tubular scaffold assembly maintained against the inner wall of the bodily lumen at the target site after the expandable element is collapsed for the period of time. The period of time may be, for instance, at least 10 seconds, at least 30 seconds, at least 1 minute, at least 2 minutes, at least 3 minutes, at least 4 minutes, at least 5 minutes, at least 10 minutes, at least 15 minutes, at least 20 minutes, at least 25 minutes, at least 30 minutes, or at least an hour. A therapeutic or diagnostic agent may be introduced to the target region and the target region may be allowed to bathe in or to absorb the therapeutic or diagnostic agent while the stent-like tubular scaffold assembly is maintained against the inner wall of the bodily lumen for the period of time. Further interventions, such as applying electrical pulses for electroporation to promote cellular uptake of the agents as described below and herein, may be applied as well. The external surface of the temporary scaffold may also comprise one or more scoring elements to score the target region and promote intake of the diagnostic or therapeutic agent. Scoring elements are described, for example, by U.S. patent application Ser. No. 14/080,917, which application is incorporated herein by reference.

The expandable element of the catheter may comprise an inflatable balloon such as a drug-eluting balloon (DEB).

Further, a proximal occluder may be positioned proximal of the expandable element and/or a distal occluder may be positioned distal of the expandable element. The proximal and/or distal occlusion element may be expanded at the target site to at least partially occlude the target site, such as when the target site is being bathed with the diagnostic of therapeutic agent.

The stent-like tubular scaffold assembly may be collapsed by actuating the elongate outer shaft of the catheter to collapse the stent-like tubular scaffold assembly. For example, a portion of the elongate outer shaft of the catheter may be retracted relative to a distal end of the elongate inner shaft, the proximal end of the stent-like tubular scaffold assembly being affixed to the portion of the elongate outer shaft of the catheter and a distal end of the stent-like tubular scaffold assembly being affixed to the distal end of the elongate inner shaft of the catheter.

Aspects of the present disclosure may also provide stent-like tubular scaffold assemblies for treating a target region with a bodily lumen. An assembly may comprise a stent-like tubular scaffold and one or more affixation members coupled thereto. The stent-like tubular scaffold may be configured to be advancable over an elongate shaft of a catheter to be positioned over an expandable member of the catheter. The stent-like tubular scaffold may have a proximal end and a distal end, and a proximal affixation member may be provided on the proximal end and a distal affixation member may be provided on the distal end to removably couple to the elongate shaft of the catheter. The affixation member(s) may comprise ring-like structure(s), elastic band(s), ratchet mechanism(s), ridge(s) and barb-like projection(s), to name a few, as described above and herein. The stent-like tubular scaffold may provide any of the advantages discussed above and herein, such as providing reduced focal points of expansion, reducing undesired trauma, facilitating more gradual expansion, limiting expansion diameter, etc.

Other features, aspects and advantages according to many embodiments can be seen on review the figures the detailed description, and the claims which follow.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the inventions of the present disclosure are utilized, and the accompanying drawings which are as follows.

FIGS. 1-10 disclose conventional blood vessel occlusion structures having expandable elements and their methods of use, as described in co-invented U.S. Pat. No. 6,238,412.

FIG. 1 is a mechanical schematic showing an occlusion and dilation catheter device fully deployed in a Dacron® graft used in hemodialysis. FIG. 1 shows the blocking element at the distal end of the catheter in its radially expanded state and the occlusion engaging element at the distal end of the support wire in its radially expanded state. It is important to note that the proximal blocking element may take a variety of shapes as may be required for the particular application. A preferred shape is likely to be a funnel shape where the larger diameter is distal to the lesser diameter that is proximal on the proximal blocking element. This funnel shape can allow the obstruction to be more easily accepted into the catheter due to the pull/push of the engaging element, aspiration or both.

FIG. 2 is a side view of the distal portion of a support wire of the device of FIG. 1 with a braided occlusion engaging element in its radial compressed state. This is the state where the support wire and engaging element can be inserted through the occlusion that is to be removed.

FIG. 3 is a side view of the braided occlusion engaging element of FIG. 2 in its radially expanded state, which is the state shown in FIG. 1.

FIG. 4 is a perspective view of a multi-wing malecot type blocking element at the distal end of the catheter in its radially expanded state, which is the state shown in FIG. 1. It should be noted that the scale of the FIG. 4 catheter device may be much reduced compared to the scale of the occlusion removal wire and braided element shown in FIGS. 2 and 3.

FIGS. 5-8 show an interventional method.

FIG. 5 is a side sectional view showing the catheter and dilator of the FIG. 1 with a ferrule at the distal tip of the guide wire in a passageway having an occlusion that is to be removed.

FIG. 6 is a side sectional view of the next step after FIG. 5 in which the dilator is being removed thereby causing the malecot type blocking mechanism to become expanded by virtue of pressure against the distal end of the catheter tip of the dilator.

FIG. 7 is a side sectional view of the next step after FIG. 6 in which the support wire together with the braided occlusion removal element in its radially compressed state (the state shown in FIG. 2) is inserted through the catheter and through the occlusion to be removed.

FIG. 8 is a side sectional view of the next step after FIG. 7 in which the braided occlusion removal element has been expanded and is being pulled in a proximal direction thereby forcing the occlusion into the catheter for removal with or without aspiration.

FIG. 9 is a perspective view of the multi-wing malecot type blocking element at the distal end of the catheter in its radially expanded state.

FIG. 10 is a side sectional view of the shape of the expansion resulting from the malecot type blocking element shown in FIG. 9.

FIGS. 11-20 illustrate examples of an interventional procedure and device, according to many embodiments.

FIG. 11 is a side sectional view of a catheter assembly with a balloon expanded at a target site, according to many embodiments.

FIGS. 12-17 show the various sequential steps in the use of the catheter assembly of FIG. 11.

FIG. 12 is a side sectional view of the catheter assembly of FIG. 11 positioned near a target site for treatment.

FIG. 13 is a side sectional view of the catheter assembly of FIG. 11 having its proximal occluder expanded.

FIG. 14 is a side sectional view of the catheter assembly of FIG. 11 having its proximal occluder expanded and distal occluder and dilation element advanced through a lumen of the catheter assembly beyond the target site.

FIG. 15 is a side sectional view of the catheter assembly of FIG. 11 having its proximal and distal occluders expanded.

FIG. 16 is a side sectional view of the catheter assembly of FIG. 11 having its proximal and distal occluders expanded and the dilation element partially expanded.

FIG. 17 is a side sectional view of the catheter assembly of FIG. 11 having its distal occluders expanded and the dilation element partially expanded.

FIGS. 18-20 show another example of a catheter assembly in which a removable, expandable braid, acting as a stent like structure, is positioned over the balloon, according to many embodiments.

FIG. 18 is a side view of a catheter assembly with the balloon and the braided stent-like structure both in expanded states.

FIG. 19 is a side view of the balloon and the braided stent-like structure of FIG. 18 in a collapsed state and an expanded state, respectively.

FIG. 20 is a side view of the balloon and the braided stent-like structure of FIG. 18 both in their collapsed states.

FIG. 21 is a schematic illustrating an electroporation catheter assembly, according to many embodiments.

FIG. 22 is a side view of a balloon catheter assembly in which a temporary braid scaffold element, which may include the braid scaffold element and an outer shaft (or ring-like fixation element), is connected to the catheter body, which may include an inner shaft and balloon, with the balloon in a collapsed state, according to many embodiments.

FIG. 23 is a side view of the balloon catheter assembly of FIG. 22 with the braid expanded and the balloon collapsed.

FIG. 24 is a side view of the balloon catheter assembly of FIG. 22 with the braid and balloon expanded.

FIGS. 25A, 25B, 25C, and 25D show cross-sectional views of an artery in which a conventional method of angioplasty is being performed.

FIGS. 26A, 26B, 26C, and 26D show cross-sectional views of an artery in which a novel method of angioplasty is being performed, according to many embodiments.

FIG. 27A shows a side view of a balloon catheter assembly in which a temporary braid scaffold element, which may include the braid scaffold element and an outer shaft (or ring-like fixation element), is connected to the catheter body, which may include an inner shaft and a balloon, with the balloon in a collapsed state and the braid scaffold element in an expanded state.

FIGS. 27B and 27C show sectional side views of the balloon catheter assembly and temporary braid scaffold element of FIG. 27A.

### DETAILED DESCRIPTION

The following description will typically be with reference to specific structural embodiments and methods. It is to be understood that there is no intention to limit the scope of the present disclosure and inventions thereof to the specifically disclosed embodiments and methods but that the present disclosure and inventions thereof may be practiced using other features, elements, methods and embodiments. Preferred embodiments are described to illustrate some of the features of the inventions of the present disclosure, not to limit their scope, which is defined by the claims. Those of ordinary skill in the art will recognize a variety of equivalent variations on the description that follows. Like elements in various embodiments are commonly referred to with like reference numerals.

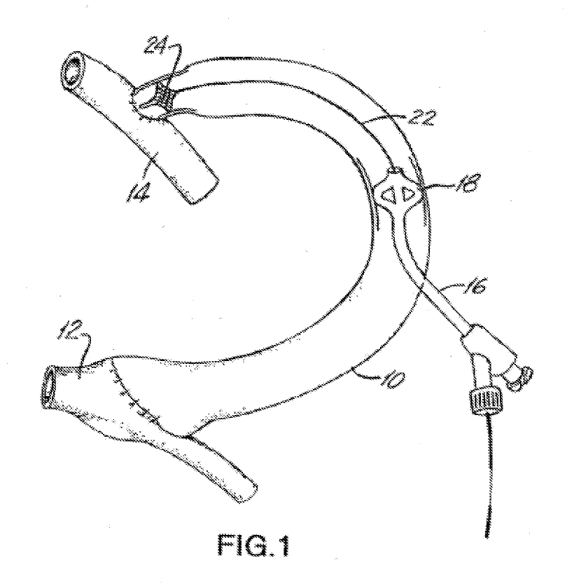

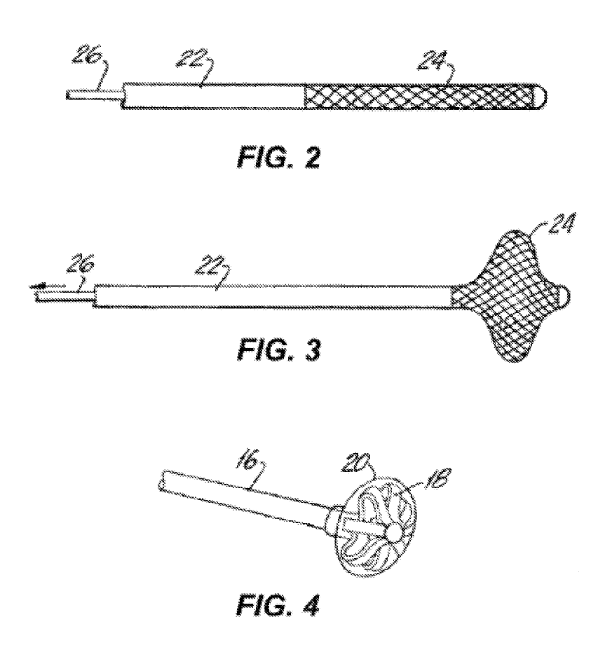

FIG. 1 shows a typical synthetic graft 10 used in hemodialysis. The graft may extend between a vein 12 and an artery 14. The graft 10 may be about thirty centimeters long with an inner diameter (I.D.) of 6 or 7 millimeters. A catheter 16 may be inserted through the wall of the graft or vessel. Typically, the catheter may have an outside diameter (O.D.) of 2.7 mm and an inner diameter (LD.) of 2.3 mm. A malecot type expansion device 18 may be covered with a membrane 20 (see e.g., FIG. 4). When expanded, the membrane 20 may serve to block the annular space between the outside wall of the catheter 16 and the graft 10. A support wire 22 for a braided removal mechanism 24 may typically have an outside diameter of about one mm and has an internal actuator rod 26 (see e.g., FIG. 2) of approximately 0.5 mm. Because of the simplicity of the design, this outside diameter could be smaller than 0.5 mm. In FIG. 1, the malecot type blocking device 18 and the braided removal device 24 are both shown in their expanded state and are positioned so that retrograde or proximal movement of the support wire 22 will pull the braided element in a proximal direction to push out whatever coagulated blood is between the braided device 18 and the distal end of the catheter into the catheter opening where it can be aspirated; thereby clearing the blockage in the graft or other vessel.

In an example, the structure of FIG. 1, which has been partly tested, was designed for use in a hemodialysis graft 10 having an I.D. of approximately six to seven mm. In that test case, the catheter 16 had an 8 French O.D. (2.7 mm) and a 7 French I.D. (2.3 mm). The support wire 22 may be a fairly standard movable core guide wire of 35 mils (that is, 0.35 inches, which is slightly less than 1 mm). The actuator rod 26 in the support wire may be approximately 15 mils and thus slightly under 0.5 mm. The braided element 24 may have an insertion diameter that is approximately one mm and expands to cover the seven mm diameter of the graft. In order to achieve this seven fold increase in diameter, the braided element may have a length of 11 to 13 mm. Thus, the catheter may have an annulus of about 2.3 mm around the support wire, through which annulus the blood occlusion is aspirated.

FIGS. 2 and 3 illustrate the support wire 22 and braided element 24 which constitute the occlusion engaging element that is moved proximal to push the occlusion into the catheter for removal. A preferred occlusion engaging element 24 may be a braided element. The braided material may have a stiffness such that it will not collapse or fold under the pressure of the occlusion when this engaging element is being moved proximally. Yet the filaments that form the braid may be flexible enough to be moved between the two states as shown in FIGS. 2 and 3. Materials from polyester to stainless steel can be used.

The distal tip of the braided element 24 may be connected to the distal tip of the actuator rod 26. The proximal edge of the braided element 24 may be bonded to the distal end of the support wire 22. Thus, when the actuator rod 26 is pushed in a distal direction relative to the wire 22, the braided device may be forced into its collapsed state shown in FIG. 2 and may be available to be pushed through the catheter and through or around the occlusion which is to be removed. When this engaging element 24 has been fully inserted, the actuator rod 26 may be moved in a proximal direction causing the braided element 24 to take the expanded position such as that shown in FIG. 3 so that subsequent movement of the entire support wire 22 may cause the braided element to move against the occlusion and push the occlusion into the distal end of the catheter. In some circumstances, the braided element 24 may be left as a braid with openings because the portions of the occlusion which may pass through the openings may be sufficiently smaller liquids so that they do not have to be removed. In other circumstances, it might be desirable to cover the braided element 24 with a membrane or film so that it becomes substantially impermeable. Further, the membrane or film covering the engaging element will be helpful in preventing trauma to the inner walls of native tissue. Even further, this membrane may be helpful in optimizing the physical characteristics of the engaging element.

With reference to FIG. 1, it might be noted that when the braided element is pushed all the way down to one end of the graft 10, as shown in FIG. 1, and then expanded it will be expanding against a portion of the wall of the graft that is smaller than the bulk of the graft. However, as the support wire 22 is pulled to move the braided occlusion removal element, the proximally, the braided occlusion element may ride on the wall of the graft and may expand as the wall of the graft expands as long as tension is maintained on the actuator rod 26.

There may be applications where the passageway involved may be a tissue passageway such as a blood vessel or other channel within the body, where this braided element 24 is expanded to nearly the diameter of the vessel so that when it is moved to push out an occlusion, it may avoid trauma to the wall of the vessel. Further, the membrane on the expanding element may aid in decreasing the trauma to native vessels as described above. In such a case, the engaging element (and the blocking element) may be used only as a seal so that the obstruction may be removed or otherwise obliterated. This seal can allow the rest of the vessel to be uncontaminated and provides for a closed system for irrigation and/or aspiration and subsequent obliteration or removal of the obstruction.

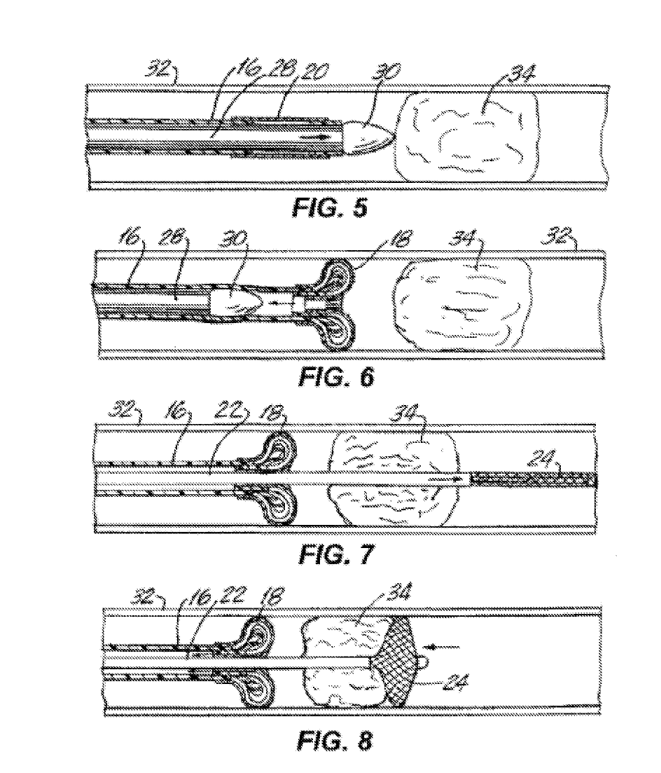

FIG. 4 illustrates the catheter 16 with the malecot 18 in an expanded state on the distal end of the catheter. A membrane 20 can normally be used in order to provide a complete blocking or sealing function. Further, the membrane 20 may aid in locking the blocking element in a particular shape. This malecot type element may be created by making longitudinal slits in the sidewall of the catheter (or an attachment bonded thereto) thereby creating links or wings that will expand when the distal end of the catheter is pushed in a proximal direction. The appropriate pushing of the proximal end of the catheter can be achieved, as shown in FIG. 5, by a ferrule 30 which is a standard tip on a standard dilator 28. Alternatively, the dilator 28 may be a guide wire (which is usually much longer and flexible than a dilator) for remote obstruction removal. In such an application, the guide wire may have a ferrule type mechanism that would act like the ferrule on the dilator. In this instance, the guide wire (with ferrule) may be inserted into the vessel to the obstruction. The catheter may then be pushed along the guide wire until it reached the ferrule which would normally be located near the distal end of the guide wire. At this point, the wire may be pulled back, the ferrule would butt against the catheter and force out the blocking sealing element. The engaging element may be used with this blocking element and it could even be the ferruled wire as well.

It should be noted that the retention catheter described in U.S. Pat. No. 3,799,172 issued on Mar. 26, 1974 to Roman Szpur illustrates a structure that is similar to the malecot type device 18 illustrated in FIG. 4; although in that patent it is used as a retention device whereas here it is used as a blocking element.

This blocking element 18 is often called a malecot in the industry. It should be understood herein that the term malecot is used to refer in general to this type of multi-wing device.

More specifically, as shown in FIG. 5, the catheter 16 together with a dilator 28 having an expanded tip 30 which may comprise a ferrule is inserted into a vessel 32 such as the graft shown in FIG. 1. The catheter 16 and dilator 28 may be inserted close to the occlusion 34 and then the dilator 28 is removed. Proximal motion of the dilator 28 may cause the tip 30 to contact the distal end of the catheter 16 forcing the distal end of the catheter to put pressure on the malecot wings creating the expansion shown in FIG. 6 (and also schematically shown in FIG. 1). Once this expansion has occurred, the dilator with its tip can be removed from the catheter (as shown in FIG. 6).

What then occurs is shown in FIGS. 7 and 8. As shown in FIG. 7, the support wire 22 with its braided removal element 24 may be inserted in the collapsed state so that it passes through or around the occlusion 34. It should be noted that the support wire 24 may be inserted prior to the blocking catheter being inserted or after the catheter is inserted (the latter of which is illustrated in FIGS. 7 and 8). Most of the occlusions to which the device of FIGS. 1-8 is directed, such as congealed blood in a graft, may permit a support wire 22 to pass through it because the consistency is that of viscous material which can be readily penetrated. Alternatively, if the occlusion is a non-viscous material such as a stone, plaque, emboli, foreign body, etc. the support wire 22 may be small enough to be passed around the occlusion. Once the braided element 24 is on the distal side of the occlusion 34, the actuator rod 26 can be pulled, creating the expanded state for the braided device. Accordingly, distal movement of the entire support wire may cause the expanded braided device to move against the occlusion and force it into the catheter for removal with or without aspiration. When removal of obstructions that are located some distance away from the point of access into the body, such as the carotid artery via a groin access, the wire 22 may likely be inserted first. In this case, the support are 22 with its expanding element 24 may be used as a guide wire to guide the catheter to the preferred location. Of further import is that the blocking element and the engaging element may be used without any relative motion once deployed. Such is the case when irrigation and/or aspiration are used for the obstruction removal. In this case, the two elements can be used as seals against the tubular inner walls on both sides of the obstruction whereby the obstruction is removed from that sealed space with the use of aspiration, irrigation, or both. Further other means of obliterating the obstruction within this sealed space may be employed. Some of those means are, but are not limited to the addition of dissolving agents, delivery of energy such as ultrasound, laser or light energy, hydraulic energy and the like.

Other Comments

An important consideration of the devices described herein may be that the support wire with its expanding element can be fabricated with a very small diameter. This can be important because it allows an optimally large annular space between the wire and the inside of the catheter for maximum obstruction removal. Previous engaging elements have been used that use a balloon for the engaging element. This balloon design may require a larger shaft diameter than that of the present devices. Hence, in these previous devices the annular space is not maximized as in the present balloon. The term wire may be used to refer to the support portion of the removal device. The material of the wire need not necessarily be metal. Further, it may be desirable to use a ‘double’ engaging element (i.e., two braided or malecot expanding elements separated a distance appropriate to entrap the occlusion) in the case, for example, where the occlusion is desired to be trapped in the vessel. The term wire may be used herein to refer to a dual element device having a shell component and a core or mandrel component which are longitudinally moveable relative to one another so as to be able to place the braided occlusion engaging element into its small diameter insertion state and its large diameter occlusion removal state.

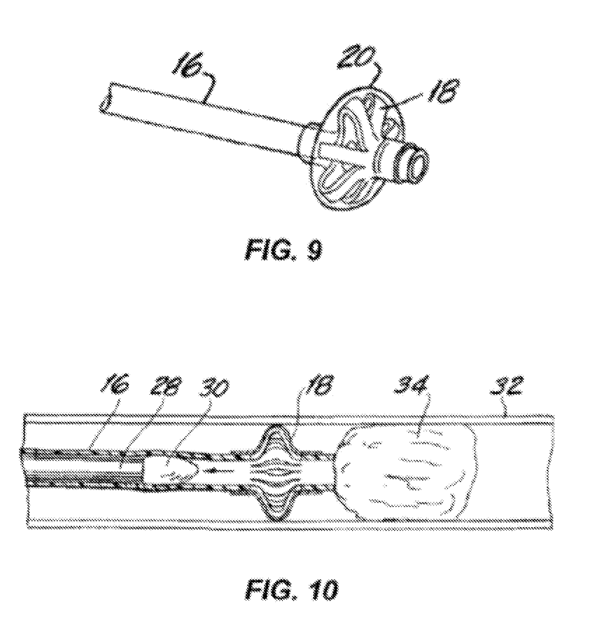

Although the blocking element is described as a malecot type of device, it should be understood that the blocking element may be designed in various fashions which are known in the art. See, for example, FIGS. 9 and 10. As another example, an appropriately designed braid arrangement could be used as the blocking element. In that case, the catheter may have to be a dual wall catheter in which the inner and outer annular walls are able to move relative to one another in a longitudinal direction so as to place the braid used as a blocking element in its small diameter insertion state and its large diameter blocking state. Alternatively, it may be a single wall similar in design to the malecot style blocking element described previously.

It should be further understood that there might be a situation in which the blocking element or even the occlusion engaging element would be provided to the physician in a normal expanded state so that when the device is deployed, it would, through plastic memory or elastic memory, automatically snap into its expanded state.

Discussion of Method for Treating a Target Site in a Vascular Body Channel

The above-described structure and methods provide a good background for the following description, which includes novel systems, devices, and methods according to many embodiments. Corresponding structures can be referred to with corresponding reference numeral, such as support wire 22/support wire 122, and occlusion 34/occlusion 134.

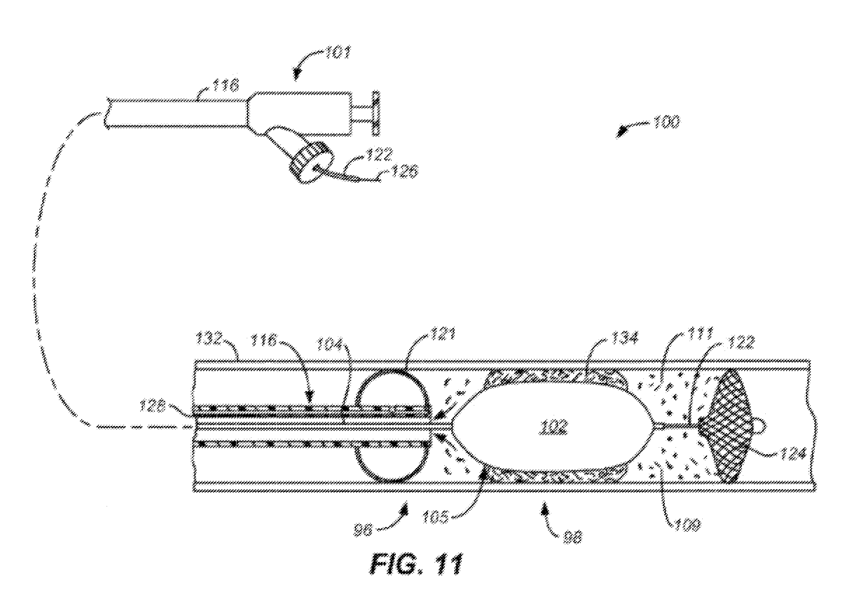

FIG. 11 illustrates a catheter assembly 100 including a proximal end portion 101, from which proximal occluder catheter shaft 116 extends and passes into blood vessel 132, and a distal end portion 96 at a target site 98 within blood vessel 132. Distal occluder 124 may be positioned at a location distal of target site 98 while balloon type proximal occluder 121 may be positioned at a location proximal of the target site to define a region 109 therebetween. Occluders of types other than those illustrated as proximal and distal occluders 121, 124, such as malecot type occluders, can also be used. However, the annular balloon type of proximal occluder 121 illustrated may be presently preferred for its simplicity of construction and lower cost. Catheter assembly 100 may also include a balloon assembly 105 comprising a balloon catheter shaft 104 passing through proximal occluder catheter shaft 116 with a balloon 102 at its distal end. Support wire 122, with an actuator 126 passing therethrough, may extend from distal occluder 124 and may pass through balloon catheter shaft 104. Balloon 102 is shown in an expanded state pressing against occlusion 134. If desired, balloon 102 could be a drug eluting or drug coated balloon. FIG. 11 also shows an injected agent 111 within region 109. Agent 111 may include various types of therapeutic and/or diagnostic agents, such as paclitaxel, sirolimus, other anti-proliferative drugs, contrast agent, thrombolytic agent, agents to dissolve the obstruction, agents to change a vulnerable plaque to a non-vulnerable plaque and the like. As discussed in more detail below, agent 111 may act on the occlusion 134 and the inside surface of a vessel 132 at the target site 98 during the intervention, in this example by balloon 102.

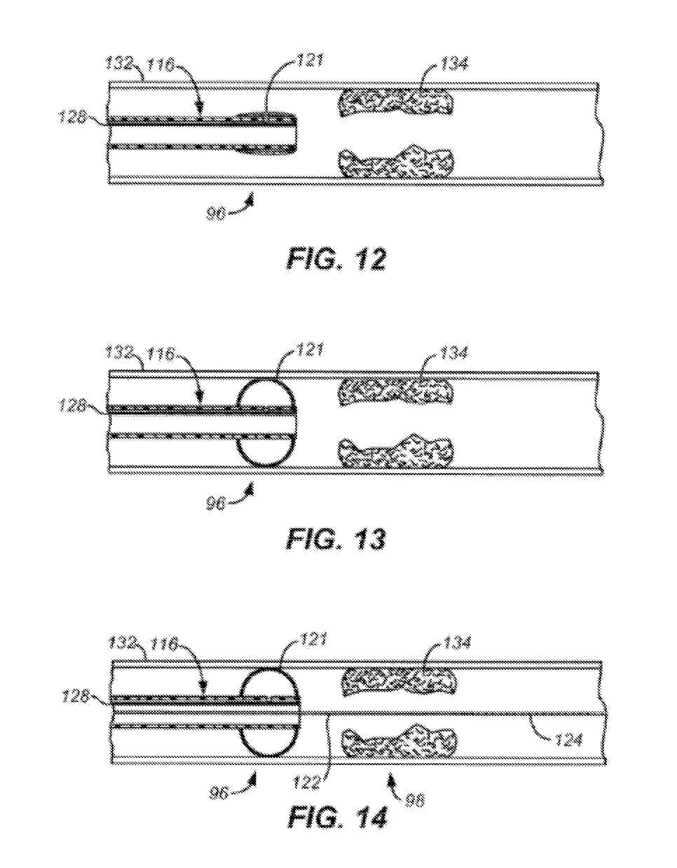

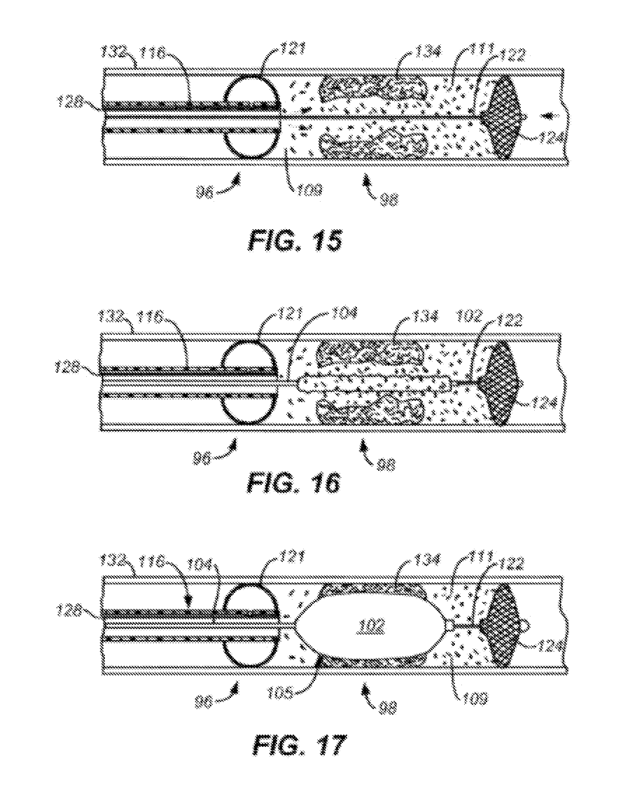

FIGS. 12-14 show the initial steps during the use of catheter assembly 100, according to many embodiments. These steps may correspond at least partially to FIGS. 5-7, discussed above, with the exception that occlusion 134 does not totally block blood vessel 132. FIG. 15 may be similar to FIG. 8 but also shows the introduction of injected agent 111 into region 109 between proximal occluder 121 and support wire 122. In some examples region 109 is aspirated through catheter shaft 116 prior to injecting agent 111. The use of proximal and distal occluders 121, 124 may concentrate agent 111 at and around occlusion 134 at target site 98. FIG. 16 shows balloon catheter shaft 104, with a balloon 102 at its distal end, inserted over support wire 122 until balloon 102, in its deflated state, is positioned at occlusion 134. FIG. 17 shows balloon 102 expanded against occlusion 134. Balloon 102 can be then deflated, back to the state of FIG. 16, followed by the removal of balloon assembly 105 to the condition of FIG. 15. Region 109 can then be aspirated to remove material from the region; the aspiration may be in conjunction with pulling distal occluder 124 proximally at least part of the way towards proximal occluder 121 and/or partial collapse of distal occluder 124 to permit retrograde blood flow past the distal occluder 124 and into region 109. Alternatively or in combination, the contents of region 109 may be allowed to flow downstream as the total dose administered would likely not be harmful to the patient. After aspiration of region 109 is complete, distal occluder 124 can be collapsed to the condition of FIG. 14 and pulled back into catheter shaft 116. Proximal occluder 121 can be collapsed by deflating balloon 102 through balloon catheter shaft 104.

The entire time balloon 102 is operating on occlusion 134, or some other intervention is being conducted at the target site 98, agent 111 may be present to bathe target site 98, including occlusion 134 and the inner wall of blood vessel 132 between occluders 121 and 124. Having the agent 111 be present to bathe the target site 98 may be extremely important because both the intervention, such as with angioplasty balloon 102, and the injected agent therapy are generally conducted essentially simultaneously without the need for removal and replacement of catheters and interventional tools.

In some examples, the proximal and distal occluders 121, and 124 are maintained in place to maintain agent 111 at target site 98 for a period of time, such as several minutes to hours, after balloon 102 has been collapsed. In some situations, more than one target site 98 may be treated through: the placement of occluders 121, 124 in contracted states, moving the occluders to a new target site, re-expanding the occluders to their expanded states, followed by injecting an agent 111 into the newly created region 109, and performing an intervention at the target site, typically using a balloon 102.

Ever since stents were introduced in the 1980's, investigators have searched for devices and methods to provide temporary support to the vascular wall without leaving a stent, which can never be removed, in the vessel forever. Bare metal stents have an unacceptable restenosis rates, and drug eluting stents, while having a moderately acceptable restenosis rate, are extremely expensive, have long term sequelae such as late stent thrombosis, and patients must stay on costly and potentially dangerous platelet inhibitor and other drugs for one year to life. Biodegradable and bioabsorbable stents have been proposed and produced, but they are less effective than either bare metal stents or drug eluting stents.

One particular use of the devices of the present disclosure may be to utilize part or all of the system before a bare metal stent (BMS) delivery. Drug eluting stents (DES) can deliver the drug to only a small portion of the vessel wall that is stented because of the spaces between the drug eluting stent struts. Utilizing the current devices with the agent injected into the closed space 109 before expansion of a BMS would bathe 100% of the vessel wall and still have the stent present to counteract elastic recoil, if it did occur, remodeling of the vessel, dissections, and other problems associated with vascular interventions. The BMS could be used with the proximal and distal occluders primarily. Alternatively or in combination, the temporary balloon stent apparatus could be utilized with the occluders and the agent between them as outlined below. If there was an unsatisfactory result after treatment with the entire system of occluders, agent, and temporary balloon stent, then the BMS may be deployed as a “bail-out” procedure. The agent may or may not be reapplied, having already been utilized before the aforementioned temporary stent application.

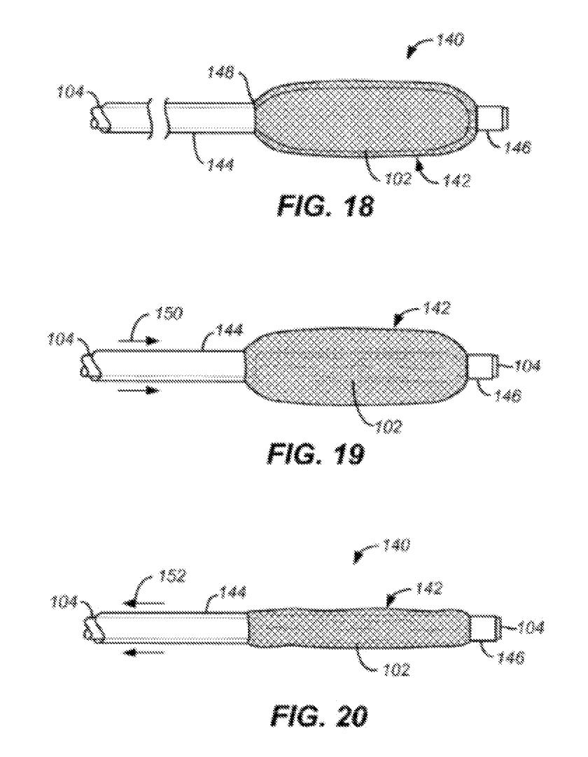

The prior art does not address a removable balloon stent apparatus that dilates the plaque and supports the wall after plaque dilatation. Lashinski et al. in U.S. Pat. No. 6,773,519 describe a stent like device which is deployed and then removed, and describes a removable coupler which is part of the device, but not a removable stent. Tsugita in U.S. Pat. No. 6,652,505 describes a guided filter which may be used to deliver a stent and removed, but not a removable stent. Kahmann in U.S. Pat. No. 5,879,380 describes a device and method for relining a section of blood vessel that has been injured or removed, not a device to both dilate the lesion and prevent elastic recoil as does the example of the present devices discussed below with reference to FIGS. 18-20.

Further examples of the devices of the present disclosure may be described with reference to FIGS. 18-20. According to many embodiments, the occlusion 134 may be dilated and elastic recoil may be inhibited by providing temporary stenting. Balloon assembly 140 may include a balloon catheter shaft 104 with a balloon 102 at its distal end and an actuator sleeve 144 surrounding balloon catheter shaft 104. A radially expandable braid 142 can be positioned over balloon 102. Balloon 102 and braid 142 are shown expanded in FIG. 18. The distal end 146 of braid 142 may be secured to the distal end of balloon catheter shaft 104 while the proximal end 148 of braid 142 may be secured to the distal end of actuator sleeve 144. Therefore, braid 142, although a stent-like structure, may be a nonremovable part of balloon assembly 140 and can be removed from the patient following the procedure.

FIG. 19 shows balloon 102 in collapsed state. By moving the actuator sleeve 144 distally in the direction of arrows 150, the braid 142 can become expanded over the collapsed balloon 102, as shown in FIG. 19, and can stay expanded when balloon 102 is deflated and collapsed. The braid 142 can be fixed to the catheter shaft 104 distally, but not to the balloon 102. It is in this expanded state of braid 142 and collapsed state of balloon 102 that the braid can act as a stent like structure and allow blood flow to be restored.

In FIG. 20, by moving the actuator sleeve 44 proximally in the direction of arrows 152, the braid 142 can be contracted against the deflated and collapsed balloon 102, and may even help lower the profile of the collapsed balloon. It is in this contracted state that balloon assembly 140 may be inserted and removed.

Balloon assembly 140 may be used by itself, that is, not as a replacement for balloon assembly 105 of catheter assembly 100 of FIG. 11. However, by using balloon assembly 140 as a part of catheter assembly 100 additional advantages can be achieved. Four separate but complementary actions can be achieved relative to the inside surface of blood vessel 132 and occlusion 134: 1) It can provide a time proven balloon action to effectively dilate the occlusion, 2) It can provide a mesh braid over the balloon to more evenly apply stresses on the plaque and thus cause less dissection and injury, 3) The braid, acting independently of the balloon, can act as a transient, removable stent to lessen acute elastic recoil, and 4) When combined with a drug delivery, it can inhibit restenosis. Currently, there are several companies in various stages of development and commercialization of drug eluting balloons. However, these devices generally do not possess the mechanical advantages of the present devices, i.e., the braid to create crevices that allow the plaque to be more homogeneously compressed at lower pressures with less injury, and the ability of the braid to be used as a transient, temporary, or removable stent to reduce the incidence of acute elastic recoil, and acting in concert with the agent to prevent restenosis. Hence, combining the mesh braid with a drug eluting or drug coated balloon may be an optimal treatment strategy.

Because the braid 142 is not attached to the balloon surface, it can act independently of the balloon 102. It is normally expanded with the balloon 102, but when the balloon 102 is contracted or collapsed to allow for distal blood flow to recommence, the braid 142 can be locked into an expanded configuration by manipulating catheter shaft 104 and actuator sleeve 144 with the fingers of the user. It is proposed that by leaving the braid expanded for several minutes while blood flow is restored distally, the smooth muscle may accommodate the stretch of the angioplasty. This may well diminish the incidence of acute elastic recoil, one of the major acute problems of POBA. In fact, prolonged expansion of the vessel has just this effect; however the time that a balloon can be left expanded is limited as ischemia will develop.

The sum of these advantages, i.e., the mechanical advantages of the braid in dilating the plaque with less pressure, less dissection, and less injury along with the temporary stent usage further combined with drug elution to inhibit restenosis is expected to significantly improve patient outcomes.

The present devices may have the potential to dramatically improve the results of POBA and the potential to improve the results of and replace DES in many cases, especially due to the ability to block the effects of recoil. Such cases may include patients with in-stent restenosis, bifurcation lesions, and small vessels lesions. DES will likely remain a dominant strategy in treating many lesions and there will likely always be a need for stenting, atherectomy and other complex treatments; but clearly if feasibility is shown, the present devices could become the treatment of choice for most angioplasty procedures. In those cases in which it may not achieve optimal results, BMS (or even DES) may then be utilized.

The present devices may occlude the lumen with a device component that will allow the angioplasty catheter shaft 104 to pass through it, and by occluding the distal aspect of the vascular channel to be perfused with the agent, the angioplasty balloon 102 and/or stent delivery balloon assembly 140 may be placed through the proximal occluder catheter shaft 116 and over the support wire 122 of the distal occluder device, the drug infused and the angioplasty and/or stent delivery can take place while the drug is present. This can allow the pressure of the angioplasty balloon 102 and/or stent delivery balloon assembly 140 to force the drug into the vessel wall while the plaque/vessel is being dilated. The drug may be delivered during the procedure and before platelet adhesion would prevent some of the drug from accessing the vessel wall as in the case of existing prior art. The presence of the drug while the action on the plaque or vessel is taking place may deliver more drug to the vessel wall than just passively bathing the vessel after the intervention.

The procedure could take several forms but one method would be to perform an angiogram to identify the lesion to be treated at the target site 98. After the lesion is identified, a diagnostic catheter may be advanced beyond the occlusion 134 and the distal occluder 124 may be deployed, which is support wire 122 and pull wire 123 based. Distal occluder 124 may be essentially a mesh braid covered with an impermeable substance. The diagnostic catheter may be removed and the proximal occluder catheter shaft 116, with proximal occluder 121 at its distal end, can be inserted over the guide wire/distal occluder and the tip of the proximal occluder is positioned proximal to the lesion. The proximal occluder may be balloon based or non-balloon based. There is a mesh braid funnel catheter occluder invented by the current inventor which occludes without the use of a balloon; see U.S. Pat. No. 6,221,006, the disclosure of which is incorporated by reference. The proximal occluder 121 and then the distal occluder 124 may be activated so that compete occlusion of the vascular lumen would be achieved. The blood may be aspirated from the region 109 between the proximal and distal occluders. The agent would be injected as injected agent 111. The agent and its concentration would be determined by the physician. The agent usually would be mixed with contrast so that it would be visible under fluoroscopy. The angioplasty balloon assembly 105 or the stent delivery balloon assembly 140 device or a stent delivery device (not shown) with a BMS or DES would be placed over the support wire 122 of the distal occluder 124 and centered on occlusion 134. The angioplasty or stent delivery may then be performed within this closed system with the agent in place. The angioplasty balloon assembly 105 or stent delivery balloon assembly 140 could then be removed through the proximal occluder 121, and the agent aspirated. The distal occluder 124 may be released and further aspiration done until blood was returned insuring that all of the drug had been aspirated before releasing the proximal occluder. The proximal occluder 121 may then be released, restoring blood flow distally.

Alternatively at this point of the procedure, if a second dilatation was desired, the drug could be aspirated through the proximal occluder after the initial dilatation similar to the above procedure, but before the angioplasty balloon was removed. Similar to above, the distal occluder may be released first while still aspirating. After blood was returned in the aspiration fluid, assuring that the entire amount of drug had been aspirated, the proximal occluder may be released restoring blood flow distally. A second dilatation of the angioplasty balloon may then be performed in a standard conventional manner without any drug being present, the drug having been delivered during the first dilatation.

However, if the desire was to deliver drug during the second dilatation, then the procedure above for the first dilatation could be repeated in a slightly modified manner. There would usually be no need to remove the angioplasty balloon. The proximal occluder may be activated, followed by the distal occluder. The blood may be aspirated and the drug may be injected through the lumen of the proximal occluder, and around the shaft of the angioplasty balloon. Then the second angioplasty dilatation may take place, the drug may be aspirated, the distal occluder may be released during aspiration, and the proximal occluder may be released to restore blood flow.

If two separate lesions in the same vascular region needed to be treated, the above may be modified somewhat. After the first lesion was treated as above, the occluders, balloon and temporary balloon stent may be collapsed and moved to a second location where the procedure would be repeated without the laborious step of changing catheters and so on. This may save time and cost, as most balloon catheters cannot be withdrawn and then reinserted into the body as the balloon folds cause reinsertion to be difficult and impractical.

If balloon assembly 140 were utilized in the above procedure instead of a conventional angioplasty balloon, braid 142, acting as a temporary stent, may remain expanded against the vessel wall in a stent like manner during the first balloon inflation, between inflations, during the second balloon inflations and for a chosen period after the last balloon inflation. This action may not only effectively deliver the drug to the vessel wall, but also may provide a temporary stenting effect to the vessel wall to inhibit acute elastic recoil.

Moreover, if balloon assembly 140 were utilized it would provide less injury to the vessel wall by dilating the occlusion at lesser pressures and causing fewer dissections. Therefore, the essence of this procedure may be to create less damage to the vessel wall, prevent elastic recoil, compress the plaque efficiently, and to deliver a drug to inhibit intimal hyperplasia as a cause of restenosis.

This procedure can have many different ways of being performed as a standard angioplasty balloon, such as balloon 102, may be used, a specialty device, such as a balloon assembly 140, may be used; in addition, stent delivery devices, laser devices, cryoplasty and most any device designed for endovascular treatment of vascular disease may be used in accordance with the present invention. The present devices may differ from prior art in that a non-balloon distal occluder is preferably used in the procedure. This difference can make it possible to perform the drug perfusion and the intervention in a single step vs. the cumbersome method of having to exchange catheters and then deliver the drug after the fact, or at least after the intervention. While other components of the present devices device have been designed for the purpose of perfusing drug after angioplasty, the presence of a guide wire (support wire 122) occluder, with any type of proximal occluder that could be traversed by a catheter, makes this device a superior one as it allows the intervention to be performed while the lesion and vessel wall are being bathed by the drug or other agent. Of course, a balloon occluder may be used distally in the method described above if it contained a shaft thin enough for an inflation channel and means to allow insertion of a treatment device coaxially over the distal occluder shaft, and it is included by this mention as an alternative.

The feature of the ability to place the treatment device over the shaft of the distal occluder so that the treatment is conducted concurrently with the drug delivery can be important to the commercial success of the procedure and method of infusing a drug to inhibit restenosis as it can obviate the less than effective method of delivering the drug in a second step in an inefficient manner after the intervention, and with a good deal of pressure upon the vessel wall. Therefore, aspects of the present disclosure may relate to performing the interventional procedure while the agent is contained within the vascular space. The present disclosure may permit treatment of variable lengths of vessel with the one device vs. the fixed lengths of devices for treating vessels in prior art. If an arterial segment that is stenosed is for example, 1.0 or 2.5 cm in length, then the entire occlusion 134 can typically be treated with a single placement of proximal and distal occluders 121, 124. If the lesion is 25 cm or 50 cm or 100 cm in length, then the same device can be used to treat any of those lesions by varying the length between the proximal occluder and the distal occluder to treat the desired length as the proximal and distal occluders may not be connected by a fixed distance as in the prior art. In long lesions, the prior art devices may need to successively move the fixed distance proximal and distal occluders (usually balloons) and provide short overlaps between each segment for multiple segments and multiple treatment sessions. The method of the present disclosure may save time, obviate repeated repositioning of the prior art device and obviate the use of multiple doses of the drug or other substance.

Balloon assembly 140 may be inserted into blood vessel 132, positioned at occlusion 134, and the balloon 102 may be inflated in a standard manner. The inflation of the balloon may expand the braid 142 and this may be the usual method of expansion of the braid. More importantly may be that the lesion will be dilated successfully, probably with a lesser pressure than a conventional POBA balloon. See FIG. 18. After a first length of time chosen by the operator, typically one or two minutes, the balloon may be deflated while force is exerted on the actuator sleeve in the direction of arrows 150. See FIG. 19. This deflation may keep the braid 142 expanded against the vessel wall while the balloon 102 is contracted allowing for blood flow to be restored distally for a second length of time, usually more than 3 minutes and typically 3 to 90 minutes. The proximal occluder and the distal occluder may be collapsed after the balloon is deflated to restore flow in the vessel while the braid is expanded against the vessel wall. The balloon inflation may be repeated as many times as desired, and by keeping forward force on the actuator sleeve 144, the braid 142 will remain expanded during, between, and after balloon inflations. There may be a locking mechanism provided so that the forward force is maintained without manual pressure. Moreover, the temporary stent may be used with modalities other than drugs, such as radiofrequency, electroporation, heat, atherectomy, gene therapy, cryotherapy, electrical currents, radiation, iontophoresis, other pharmacological agents and substances, and the like.

Combining the various elements described herein, including the temporary stent to dilate the lesion at a lesser pressure with less injury to the wall and to be utilized to reduce or eliminate elastic recoil along with one or more of the other modalities, may eliminate the need for the administration of a drug agent to inhibit restenosis. Combining the drug administration with another modality listed above and the temporary stent element may even further solve many of the short and long term sequelae of vascular intervention, and may even further eliminate the need for stenting or surgery in many cases. If the dilatation of the lesion was adequate because of the proven effect of the typically wire-like temporary stent exterior to the balloon being able to dilate plaque more effectively than POBA, if the lesion was held open by the temporary stent while the drug acts upon the smooth muscle cells and to relax them preventing elastic recoil, and another modality from the list above, for example electroporation, was utilized to enhance the absorption of the drug and to act on the cells of the vascular wall independently to further inhibit restenosis, then all of the reasons to use conventional, non-temporary stents would be obviated. The problems that stents solve may be eliminated. There may be no reason to use a stent in many cases, and this would benefit the patient and the healthcare system. Stents may not only be costly, but can have long term negative consequences, including in-stent restenosis, late stent thrombosis, and the need to be placed on expensive and potentially deleterious drugs for extended periods.

In the case of electroporation, and some of the other modalities, an electrically conductive temporary stent could be used to transfer the energy or electrical pulses to the vessel wall. FIG. 21 illustrates an electroporation catheter assembly 100a which may be constructed to permit the application of electric current to the wall of blood vessel 132 to create transient pores in the cell membrane through which, for example, a drug may pass. Braid 142, acting as a temporary stent, may be connected to an external power source 160 and a computer-based controller 162 by wires 164 within the wall of external actuator sleeve 144. Controller 162 may be utilized to program the pulse duration, sequence, amplitude, voltage, amperage, and other parameters to deliver the prescribed energy or electrical pulses to the vessel wall through the temporary stent. It may also be utilized to ascertain electrical impedance or other feedback parameters so that the proper energy parameters may be programmed or prescribed. In this example, actuator 126 electrically may connect distal occluder 124 to ground 166 thereby grounding the vessel wall. Alternatively or in combination, the energy, such as in the form of electrical pulses, may be delivered through a configuration other than the temporary stent. The electroporation may be used to facilitate the delivery of a drug, but also may be used alone to create the pores in the wall of the cell without the drug being present. The cell may then be unable to recover from having these pores in the wall, and it eventually dies, in effect a type of accelerated apoptosis. Adding the additional modality to the system of occlusion elements, drug infusion, dilatation and temporary stenting described herein would add very little incremental cost, but may be necessary to reduce the restenosis rate under the 10% rate expected from the above system without the additional modality.

The medical literature demonstrates that paclitaxel acts on the cytoskeleton or microtubules within smooth muscle cells by enhancing polymerization and causes the smooth muscle cells to relax. There are other cellular effects, certainly, but the dysfunctional microtubules are thought to be reason the smooth muscle cell relaxes rather than contracts as a result to exposure to certain drugs. The temporary stent created by braid 142 combined with paclitaxel may provide enough time of prolonged distension of the vessel for the paclitaxel to act upon the cytoskeleton and microtubules so that the smooth muscle cells would not contract upon the removal of the temporary stent. Embodiments of the present disclosure can take advantage of paclitaxel or other antiproliferative drug through the use of the braid 142 acting as a temporary stent to provide this action of prolonged expansion, allowing the drug to act upon the cells so that they will not contract when the temporary stent is removed. Without the prolonged expansion, the drug may likely not have enough time to act upon the cellular components to cause the smooth muscle cell to relax. The extra time provided by the expanded temporary stent while blood is flowing through the area along with the uptake and action of the drug will likely result in diminished elastic recoil of the vessel, and better long term patency.

Also disclosed herein are methods of infusing an antiproliferative drug or other agent that acts upon the smooth muscle cells and structures within the arterial wall and prolonging the distension of the vessel with a balloon, a temporary stent or scaffolding, or other structure to reduce the incidence of elastic recoil, restenosis, and/or other effects of the intervention.

In one example, the methods may entail placing the proximal and distal occluders on each side of the lesion to create an isolated region, activating the proximal and distal occluders, injecting the drug, performing the therapeutic angioplasty intervention with a temporary stent device as has been described leaving the temporary stent expanded against the vessel wall, deflating the angioplasty balloon so blood flow could be restored subsequently, aspirating the drug along with other flowable material (or even removing it from the isolated region by releasing it downstream), deactivating the proximal and distal occluders, and removing the distal occluder. This may restore flow in the vessel, but the temporary stent may still maintain annular pressure against the vessel wall to prevent elastic recoil while the drug, having been absorbed by the smooth muscle cells, acts upon the microtubules of those smooth muscle cells to create a relaxation of these smooth muscle cells and prevent acute elastic recoil. In some examples, the drug or other agent may be allowed to contact the target site for a period of time, such as from 30 seconds to 20 minutes, before the therapeutic angioplasty intervention, or other pressure applying step, is performed. In some examples, the angioplasty balloon, or other pressure applying apparatus, may be used to apply pressure to the vessel wall from about one minute to five days. When the balloon is left in place for extended periods, it may usually be in a collapsed state to permit blood flow around it. It may be expanded only when necessary, such as to expand the lesion during angioplasty and to expand the temporary stent.

Alternatively, the above example may be modified so that instead of a temporary stent, a plain angioplasty balloon device, a stent, such as a bare metal stent or a bioresorbable or biodegradable stent which is intended not to be removed, atherectomy, or other therapeutic device is utilized. Also, the deactivated proximal and distal occluders may be left in place within the vessel while a pressure device is providing force against the vessel wall, and removed when the pressure device is removed. The temporary stent or other pressure device may typically remain in place for at least several minutes and at most for several hours to days to prevent elastic recoil. If, for example, the balloon assembly 140 of FIG. 20 is left in place for several days, balloon 102 may be collapsed to permit blood flow around it. In such a procedure, heparin or some other agent could also be administered.

Moreover, the temporary stent may be used with other modalities other than drugs, such as radiofrequency, electroporation, heat, atherectomy, gene therapy, cryotherapy, electrical currents, radiation, iontophoresis, other pharmacological agents and substances, and the like.

Other variations of temporary stenting, can be used. For example, the braid 142 may be contracted by guide wire(s) instead of the actuator sleeve 144. The braid may be contracted by moving the distal part of the braid more distally by using an engagement device instead of an actuator sheath. In other words, if the distal aspect of the braid may be engaged or attached to the distal aspect of the guide wire rather than fixed to the distal aspect of the balloon catheter as described in the preferred embodiments, then moving the guide wire distally would collapse the braid and moving the guide wire proximally would expand the braid, or at least maintain expansive pressure upon the already expanded braid.

The use of the temporary stent with drug coated balloons or drug eluting balloons merits further discussion, although the following discussion may be applied to standard angioplasty balloons or other interventional devices While it is entirely feasible to utilize the temporary stent over a drug coated balloon as an integral part of the device in which the drug coated or drug eluting balloon and the temporary stent are combined and secured together before packaging and sterilization, the temporary stent may be configured so that it is placed coaxially over the standard angioplasty balloon, drug coated balloon or drug eluting balloon at the time of the procedure by the operator. In this configuration, the drug coated balloon or any other balloon may be placed coaxially through the temporary stent. The temporary stent may comprise a single elongate member attached to the braid or other expansile member. In this instance, a preferred configuration may comprise an attachment mechanism distal to the expansile member of the temporary stent that will engage the shaft of the balloon catheter distal to the balloon. Because of the push/pull method of tensioning, expanding and collapsing the expansile portion of the temporary stent in some configurations, an attachment of the temporary stent to the balloon catheter may be needed and may be provided by any one of several attachment mechanisms including but not limited to standard compression rings, compression rings with internal barbs that penetrate the tip of the balloon catheter distal to the balloon, a suture means of attachment, a braided structure, a screw type fitting, compression fittings, bonding and the like. This distal attachment mechanism may be incorporated into the bonding or other fixation mechanism which secures the outer stent-like tubular scaffold 142 of FIG. 23 to a distal shaft section 146 of the outer apparatus. To fixably attach scaffold 142 to distal shaft section 146 and to affix the outer apparatus to the distal shaft 104 of the inner balloon catheter, fixation elements pierce the wall of element 146 and extend into the lumen of element 146 where they may engage the outer wall of element 104 may be required. Preferentially, the attachment mechanism should be used to secure the distal portion of the outer member to the distal portion of the coaxially encompassed inner member by the operator. In the case of utilizing a self-expanding expansile member of the temporary stent, some connection with the distal aspect of the balloon catheter may also be needed as well as a sheath over the self-expanding expansile member. In essence, the angioplasty balloon catheter or other interventional device becomes the “inner member” of the prior embodiment comprising the inner sheath and balloon and the new configuration comprising the outer sheath and the expansile member becomes the “outer member.” Since they are attached distally by some mechanism, the push/pull action previously described to expand and collapse the expansile member is preserved utilizing the push or pull on the shaft of the angioplasty balloon or interventional device vs. the push/pull on the coaxially placed shaft of the alternative embodiment.

There also may be a mechanism on or near the proximal hub which can secure the temporary stent apparatus to the inner balloon catheter or other interventional device. This mechanism may be a standard Thuoy Borst type compression fitting or other mechanism that secures the position of the outer shaft of the temporary stent to the coaxially placed inner shaft of the balloon catheter. The mechanism may employ a friction fit that resists free translation of the inner and outer shafts. The mechanism may allow translation of the inner and outer members when a certain amount of tension or traction is placed upon one or the other of the inner and outer sheaths so that when expansion of the balloon distends the expansile member of the temporary stent, the outermost coaxial sheath of the temporary stent will translate distally with respect to the innermost coaxial sheath of the balloon catheter. Hence, the proximal securing mechanism may place a consistent resistance on the expansion of the expansile member of the temporary stent and therefore limit the expansion of the coaxially encompassed balloon while the balloon is being inflated and may limit the extent of expansion of the balloon.

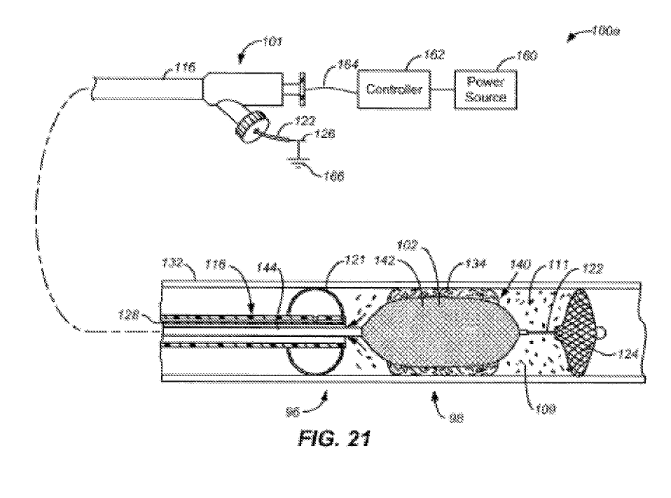

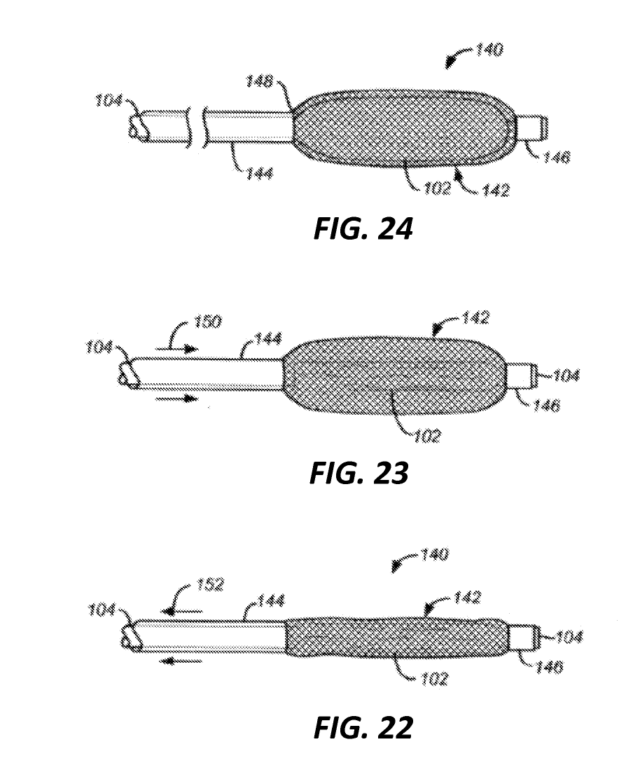

FIG. 22 is similar to FIG. 20 except the temporary stent apparatus shown may comprise only the outer sheath 144, mesh braid 142 and the connection apparatus 146 which may be completely separate from the balloon catheter assembly which is comprised of the inner sheath 104, the balloon 142 and the tip of the balloon catheter 104. To function, the balloon catheter assembly may be inserted through the outer sheath 144 of the temporary stent apparatus, through the mesh braid portion 142, so that the tip 104 of the balloon catheter apparatus extends past the connection apparatus 146 as shown in FIGS. 22-24. After insertion of the balloon catheter assembly into the temporary stent apparatus, the connection apparatus 146 may be secured to the tip 104 of the balloon catheter assembly by the appropriate method.

The method of utilization may be to obtain a standard angioplasty balloon catheter assembly which may or may not be a drug coated balloon catheter assembly and insert the said balloon catheter assembly through the shaft of the temporary stent, through the expandable portion and out the end of the temporary stent so that the balloon is placed within and enclosed by the expandable portion of the temporary stent apparatus. Then, the operator may affix the connection apparatus of the temporary stent to the distal tip of the balloon catheter assembly using one of the connection means listed above. This connection may allow the push/pull maneuver to maintain expansion of the expandable portion of the temporary stent after the balloon is collapsed and to collapse that portion for removal. The balloon may be expanded, which will expand the expandable portion of the temporary stent. Rearward traction on the outer sheath of the temporary stent may constrain the free expansion of the balloon and cause it to expand in a cylindrical pattern rather than expanding into areas of diminished resistance, which is the usual case. Alternatively or in combination, a mechanism near the proximal hub of the outer elongate member of the temporary stent may be employed to prevent the free translation of the outer elongate member and hence constrain the expansion of the balloon in a manner similar to providing rearward traction on the outer elongate member. After the balloon is fully expanded, forward pressure on the outer sheath of the temporary stent may be exerted to maintain expansion of the expandable portion for a period of time while the balloon is collapsed. Retracting the outer sheath of the temporary sheath may collapse the expandable portion for removal.

In general, the temporary stent may act in a sock like manner being placed over the drug coated balloon or other balloon catheter. This may allow the temporary stent to be utilized with any number of drug coated or other balloon catheters produced by various manufacturers.

The shaft 144 of this configuration of temporary stent may be sized so that the internal diameter accommodates the outer diameter of the shaft 104 or the balloon 102 of the selected drug coated balloon or other balloon catheter. The length of the temporary stent expansile member may be sized so that the expansile member accommodates the length and diameter of the drug coated or other balloon section of this catheter.

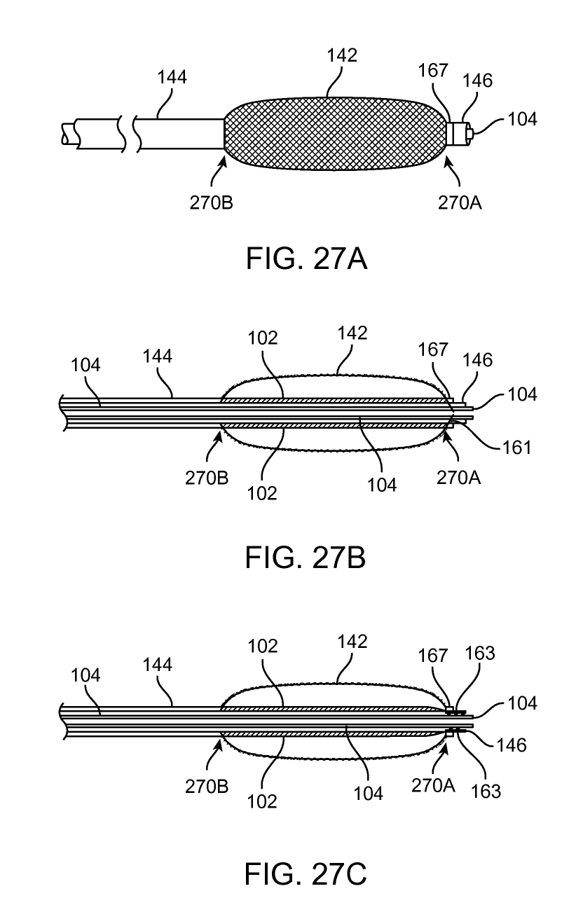

The attachment means distally that serves to secure the temporary stent to the distal shaft of the balloon catheter may comprise a friction fit means that simply slides over the distal shaft of the balloon catheter or a modified friction fit that comprises small barbs that prevent movement of the outer temporary sheath mechanism relative to the inner balloon catheter. Other mechanism of securing the temporary stent to the balloon catheter may include, but are not limited to, mechanisms that screw or lock into place, compression fittings, interference fits, magnetic attachments, or other means that secures one to the other. The attachment means distally are preferentially configured to be secured to the distal end of the coaxially encompassed balloon catheter by the user of the device at the time of use. The distal tip 146 of the outer alternative embodiment may be tapered to the outer diameter of the distal tip 104 of the balloon catheter so as to make a smooth transition. As shown in FIGS. 27B, and 27C, the ridges 163 or barb-like structures 161 may create friction with the outer surface of the elongate shaft of the expandable element catheter and/or the ridges or barb-like structures when inserted coaxially into the distal shaft 146 of the instant device, and/or may be shaped to penetrate the outer surface of the coaxially encompassed distal elongate shaft of the expandable element catheter which may prevent any translation of the inner and outer distal components. These ridges 163 or barb-like structures 161 may be angled, may be pointed, may be circumferential or non-circumferential, may be oriented in patterns including, but not limited to, a screw like pattern and may be oriented toward the center of the outer elongate shaft or to the proximal, distal, or radial aspect of the outer elongate shaft or a combination of the above.

Other configurations may not involve a balloon at all. In these configurations, the temporary stent may comprise an outer and inner sheath and an expansile member similar to the above descriptions, but instead of the expansile member being placed over a balloon, the inner sheath or shaft may be just a shaft with no balloon. This configuration may be used after an initial dilation by a POBA, drug coated balloon or other balloon and inserted separately, preferentially after the balloon dilatation, as needed to support the vessel wall because of a flow limited dissection or elastic recoil. It may be utilized in cases where “bail out” stenting is considered to further improve the post angioplasty results to avoid permanent stenting.

In the “sock like” configuration in which the temporary stent is a device separate from the balloon or other interventional device that may be enclosed by the expansile member of the temporary stent, the mechanism of action may be similar to the preferred original embodiment in which the balloon or other interventional device and the temporary stent are combined by the manufacturer before packaging and sterilization. Because of the attachment of the temporary stent distally to the distal portion of the inner balloon catheter, the balloon would expand the braid against the wall. The braid may comprise the same or different configuration as in the original preferred (non “sock like”) embodiment with varied configurations of the members of the braid, and the expansile member may be tensioned or expanded by forward motion of the outer sheath in relation to the shaft of the balloon catheter and collapsed by rearward movement of the outer sheath in relationship to the shaft of the balloon catheter. These forward or rearward positions may be secured by adjusting the proximal fixation mechanism. These relationships are demonstrated in FIGS. 23 and 24 and the actions correspond to the actions previously discussed in FIGS. 19 and 18, respectively. In FIG. 24, the balloon 102 is expanded which expands the braid 142 of the temporary stent. In FIG. 23, the mesh braid 142 may be maintained in the expanded state while the balloon 102 is collapsed by advancing the outer sheath 144 of the temporary stent apparatus in relation to the shaft 104 of the balloon catheter assembly as shown by arrows 150. It may be collapsed as in FIG. 22 by withdrawing the outer sheath 144 of the temporary stent apparatus in relation to the shaft 104 of the balloon catheter assembly as shown by arrows 152. As shown in FIGS. 27A-27C, the distal portion 146 of the braid 142 may be coupled either removably or fixedly to the inner sheath 104 at distal point 270b, and the proximal portion of the braid 142 may be coupled either removably or fixedly to the outer sheath at proximal location 270b. As shown in FIGS. 27A-27C, the distal portion 146 of the braid 142 may comprise a ring-like structure 167 to fit over the catheter shaft 104. Alternatively or in combination, the proximal portion of the braid 142 may comprise a similar ring-like structure to fit over the actuator sleeve 144. As shown in FIGS. 27A-27C, the balloon 102 is in the collapsed state and the braid 142 is in the expanded state, and the outer shaft or sheath 144 can be proximally retracted relative to the inner shaft or sheath 104 to collapse the braid 142.

In addition to use with drug coated balloons, standard POBA balloons, weeping balloons, and other balloons, the temporary stent may be used with other devices that may deliver drugs, genes, cells, cellular components, pharmaceuticals, particles, other fluids both within the lumen, to the wall of the vessel, or into the tissues surrounding the vessel. Examples may include the Mercator Bullfrog micro infusion catheter, the GENIE by Acrostak and other drug delivery devices. In fact, the sock-like temporary stent of FIGS. 22-24 may be utilized with any means of expansion, not necessarily a balloon catheter.

Even other configurations of the “sock-like” temporary stent may utilize a self-expanding stent-like device that is tethered to a shaft or other means so that it is not detached from that shaft or other means. The temporary stent structure, in this instance, may be placed over the inflatable balloon of the balloon catheter as in the prior examples, but may not be attached to the distal end of the balloon catheter at all. In these configurations, there may be a sheath which covers the self-expanding temporary stent-like device to keep it constrained or collapsed while delivering it to the site to be treated and when removing it from the body. This outer sheath may simply be withdrawn from a position over the self-expanding stent-like device to expose it and advanced over the self-expanding stent like structure to collapse and contain it. The self-expanding stent-like structure may possess any of the features previously described herein including, but not limited to, being constructed of standard stent materials and construction, braided materials, or other configurations. If constructed of braided material, the braid may be configured in any of a number of ways. The stent-like device may even comprise a drug eluting features from the stent members.

The method of using the “sock like” self-expanding temporary stent described in the preceding paragraph may entail placing the temporary stent, constrained in the outer sheath, over an angioplasty balloon catheter, a drug coated balloon catheter, a weeping balloon catheter or other balloon catheter, withdrawing the outer sheath to allow the temporary stent to expand over the balloon, inflating the balloon of the inner catheter for a certain period which further expands the self-expanding temporary stent structure, deflating the balloon while leaving the temporary stent expanded for a certain period, potentially repeating the inflation/deflation cycle once or more, and leaving the temporary stent expanded while the balloon is deflated for a certain period of time, which may vary from 1 minute to 60 minutes or longer, but preferably from 4-15 minutes. Alternatively, the self-expanding stent-like structure may be utilized subsequent to a balloon dilatation instead of concurrently as described previously

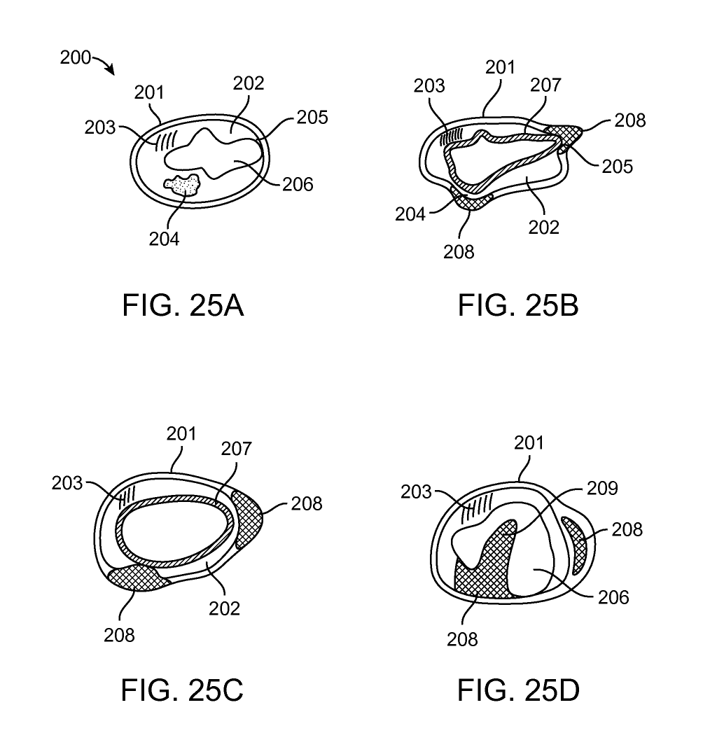

Normally, during the process of angioplasty, the angioplasty balloon expands a plaque to restore the lumen of the vessel. FIGS. 25A-25D demonstrates the angioplasty process with a standard existing angioplasty balloon. FIG. 25A demonstrates a cross section of a blood vessel 200 with a wall 201 of the blood vessel 200, a complex plaque 202 within the blood vessel which contains a dense, fibrotic area 203, a soft, non-fibrotic area 204 and an area that is relatively thin 205. The thin area 205 of the plaque allows the lumen 206 to extend nearly to the wall 201 of the blood vessel 200. In FIG. 25B, a standard angioplasty balloon 207 has been inserted and partially expanded. The expansion of the balloon 207 with fluid occurs non-uniformly as the balloon 207 expands against the areas of least resistance, at least during most of the expansion even with a non-compliant balloon in which the final expansion and shape are defined and limited. The balloon 207 preferentially expands into the soft non-fibrotic area 204 and into the relatively thin area 205. This causes areas of the vessel wall and the plaque to experience focal areas or zones of excessive pressure and abnormal horizontal, radial and torsional forces as the balloon preferentially expands into the areas which provide the least resistance because of the dimensions or thickness of the plaque or because of the consistency of the plaque. The calcific or densely fibrotic portions 203 of the plaque may be much firmer and resistant to compression than non-calcific or less fibrotic portions 204, 205 of the plaque for instance. Hence, the balloon expansion may cause an inordinate amount of expansion and resultant excessive pressure and forces to be exerted to the areas or zones that are more easily expandable. These are the areas or zones that are most subject to damage by the angioplasty procedure. Because of the excessive pressure, areas of dissection, hemorrhage and damage 208 are created during balloon expansion.

As shown in FIG. 25C, with full expansion of the balloon 207, the plaque 202 is asymmetrically compressed and the areas of damage 208 are further worsened and enlarged by the additional pressure. These areas of damage are primarily responsible for restenosis, which is the Achilles heel of angioplasty. Preventing these areas of damage would prevent restenosis in many cases. FIG. 25D demonstrates the vessel 200 after removal of the angioplasty balloon 207. The fibrotic area 203 is incompletely compressed, the areas of dissection, hemorrhage and damage 208 are further enlarged as hemorrhage continues, and a dissection flap 209 of tissue projects into the lumen 206. This dissection flap 209 may be flow limiting as it compromises the lumen 206. Further, because of the dissection, hemorrhage and damaged areas 208, the blood vessel 200 may reflexively contract and spasm even further diminishing the lumen 206. This latter condition may be referred to as elastic recoil.

Hence, it may be advantageous to prevent these focal areas of relatively excessive balloon expansion and relatively excessive pressure on the vessel wall during the expansion of the balloon. Another application of the “sock like” temporary stent that is placed over an angioplasty or other balloon, or even the standard temporary stent that may combine all components into a single device described previously, is to constrain the expansion of the angioplasty balloon during the expansion of the balloon. The discussion above and below may apply to either embodiment.

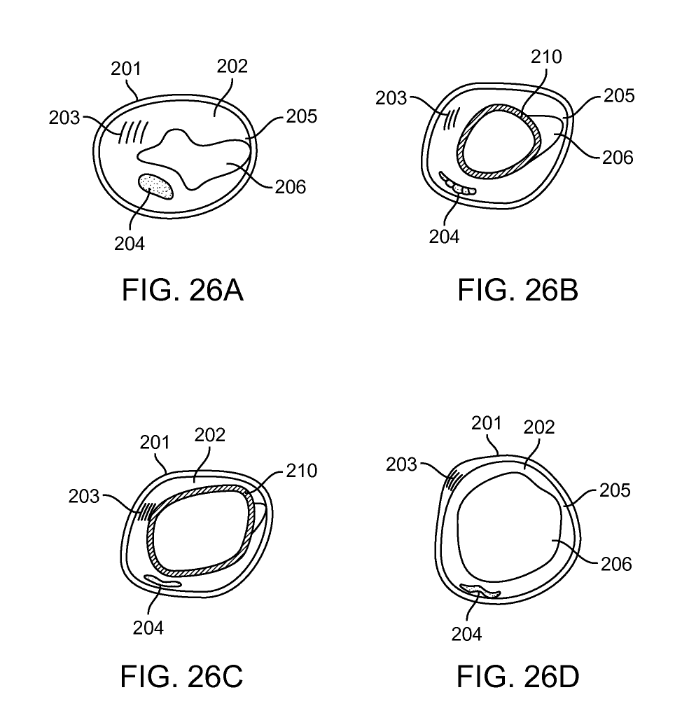

Typically, in this instance, the balloon may be a semi compliant or non-compliant balloon. The braid or stent like structure comprising the expansile portion of the device would constrain the balloon expansion by either by maintaining tension on the expansile member and limiting balloon expansion or by reaching the limits of expansion of the expansile member so that the braid or stent like expansile member will not expand further than desired. This method may be effective in limiting the damage to the blood vessel. The best results may be obtained by maintaining tension on the expansile member by utilizing the push/pull mechanism of the previously described shafts of the respective devices to prevent the expansile member from expanding beyond a certain level during the balloon expansion. The tension may be applied from the beginning of the balloon expansion so that the expanisle member constrains the balloon while expanding, in other words. This may cause the outward radial expansion and outward radial forces within the balloon during the expansion phase to be more or less uniform throughout the length of the balloon and prevents focal bulging and expansion of the balloon into an area of soft or no plaque which may exert an inordinate amount of focal pressure and stress forces on the arterial wall that may result in damage to the wall at that point. Maintaining active tension on the expansile member while the balloon is expanding may provide a more uniform cylindrical expansion of the balloon with more uniform pressures as well as limiting non-uniform longitudinal, radial, and twisting or torsional forces being transmitted to the vessel wall. The balloon shape may be continuously constrained by the tension of the outer expansile member during the critical expansion phase preventing focal area or areas of excessive expansion and pressure onto the wall of the vessel. The outer expansile member, when held in tension, may squeeze the expanding balloon and may prevent areas of relative over and under inflation and areas of relative increased and decreased pressure and forces while the balloon is expanding. Hence, the same pressures may be exerted along the length of the balloon when the balloon is minimally expanded, moderately expanded, near completely expanded, and completely expanded. This is illustrated in FIG. 26 A-26D. FIG. 26 A demonstrates the vessel 200 and the plaque 201 as previously described with areas of fibrosis 203, soft areas 204, a thin area 205 and a lumen 206. In FIG. 26B, an angioplasty balloon has been placed coaxially within and constrained by an outer expansile member 210 and has been partially inflated. The balloon/expansile member structure 210 expands symmetrically and uniformly in a cylindrical configuration rather than expanding into the areas of least resistance in the case of FIGS. 25A-25D. The fibrotic area 203, the soft area 204 and the thin area 205 of the plaque 202 are demonstrated.

In FIG. 26C, complete expansion of the balloon/expansile member apparatus 210 is demonstrated with a relative uniform compression of the plaque 202 without the areas of hemorrhage, dissection and damage in FIGS. 25A-25D. FIG. 26D demonstrates the balloon/expansile member apparatus 210 to have been removed with the resultant expanded lumen 206 of the vessel 200 and the relatively uniformly compressed plaque 202. In the instant methods of angioplasty utilizing the balloon/expansile member, significant damage to the vessel wall can be avoided and the sequalae of recoil, flow limiting dissection, and restenosis is also avoided. Hence, employing stents to treat the sequalae may also be avoided by utilizing the current methods.

By applying the active compressive forces of the expansile device, the balloon may exert the same outward radial pressure and more or less uniform radial, axial and torsional forces along its length and about its circumference when the balloon is inflated to 1 atmosphere pressure, 3 atmospheres pressure, 10 atmospheres pressure, 30 atmospheres pressure or all of the points in between, respectively. Other devices and methods to constrain a balloon only limit the expansion of the balloon during complete expansion and do not provide an “active” mechanism or method of consistent constraining force limiting the expansion during the entire balloon inflation. Hence, those devices may allow, and do allow, uneven pressures to be transmitted to the plaque and vessel wall during an inflation and hence will allow inordinate and damaging pressure to be transmitted to the vessel wall during balloon inflation and at all times other than complete inflation. The expansile braid or stent member may preferably be non-elastic, although it may be elastic, and non-deformable as the tension placed on the expansile member from the push/pull effect is the method of constraining the shape of the balloon while it is expanding.

With the balloon expansion limited, the balloon may bulge through the pores or interstices of the braid or stent like expansile member and produce focal more or less diamond shaped areas of pressure on the vessel wall and plaque to compresses the plaque more evenly and in a more uniform manner. The orientation of the braid filaments may be in a clockwise and counter clockwise direction creating the diamond like areas of balloon bulge and intersecting spiral lines of balloon constraint. The angle of crossing of these filaments may be between 15 to 75 degrees, or alternatively 105 to 165 degrees if the measurement is on the outer angle of crossing, depending on the braid or stent construction and the degree of braid or stent expansion. Preferably, the angles are 25-60 degrees. The angles may change as the braid or stent is expanded or contracted. This type of construction may occur with any or some of the configurations listed previously or may utilize large pores within the braid by varying the pic count, utilizing a non-axisymmetric mandrel during the braiding or other braid variable so that the balloon bulges though the pores of the braid somewhat. It may not allow focal areas or points of full balloon expansion and other areas are incompletely expanded because of the plaque as existing balloons do which may create uneven pressure transmission to the vessel wall with zones or areas of very high and very low pressure.

This controlled dilatation may reduce the stresses on the plaque and vessel wall which may result in reduced dissection, elastic recoil, and vascular damage with subsequent restenosis, the same complications that the temporary stent apparatus addresses by temporarily propping the vessel open. By limiting the damage with one or both of scoring elements, focal areas of more uniform pressure as just described, slow inflation times and other methods and combining these techniques with the stent like benefit of the current devices, one may obviate the use of standard permanent stents in many cases.

The braid or stent like expansile member may comprise filaments or struts that are coated with one or more of a drug, gene therapy, medicament or biologically active material and may provide energy of heat, radiofrequency, electrical, or other. Moreover, the balloon discussed may provide cooling, heating or energy transfer of some manner directed to achieving the goal of creating less damage to the vessel wall and preventing recoil, restenosis, dissection and vessel closure.

Furthermore, the active and separate control of the braid over the balloon provides a mechanism and method for collapsing the balloon after balloon expansion not provided in other devices. Typically, the angioplasty balloon in folded upon itself for insertion. Frequently, the balloon may resist assuming the lower profile pre-inflation state and may not return to the original collapsed uninflated configuration even despite the maneuvers developed by extensive research to accomplish that. This may be problematic in removing the balloon catheter with the incompletely collapsed balloon with wings protruding outward. The partially collapsed balloon may not fit through another delivery catheter or sheath or may damage the vessel when being withdrawn. Hence, there may be a need for a fully compressed deflated balloon after a therapeutic inflation, and the expansile member may accomplish this task more completely when the outer sheath is put into traction tension relative to the inner shaft collapsing and squeezing the balloon further than simply deflating the balloon will. The outer expansile member may optimally collapse the overall diameter of the balloon including the wings of the balloon by 5-50% depending on the construction and technique of folding the balloon, and preferably between 10-25% in well-constructed, properly re-folding balloons. This may be enough to ensure uneventful removal of the inflated/deflated balloon in all cases. Hence, because of the active tension on the outer expansile member, expansion of the balloon element may be limited during deflation to provide for uniform expansion and also compress the balloon element during deflation to achieve a more complete collapse of the balloon element.

As stated previously, any one of the features of the systems, devices, and methods described herein may be utilized with any one or more of the other features described herein.

Embodiments of the present disclosure may differ significantly from those described in the Ya patent discussed above in that no dissolving agent outflow bores may be used, the embodiments may not be directed to dissolving a thrombus, and any antiproliferative agent may be injected before the intervention and may be present during the therapeutic intervention, not removed before the intervention as in Ya. Any subsequent intervention or therapy (angioplasty, stent placement, and the like) may be performed after the removal of the dissolved thrombus in Ya. Moreover, the thrombus dissolving agent and the dissolved thrombus must be removed in the method of Ya, which is aimed at removing a thrombus, whereas there is typically no need to remove any antiproliferative agent when practicing the embodiments of present disclosure. The dose of the antiproliferative agent may be much lower than the systemic dose administered a patient receiving chemotherapy for treatment of a tumor.

In most embodiments disclosed in the Zadno-Azizi reference discussed above, the device is comprised of two distinct lumens, an irrigation pathway and an aspiration pathway, much different from the device and method of the current disclosure. In the single example disclosed in Zadno-Azizi in which there is only a single aspiration path, the therapy catheter must be removed for the device to function. In contrast with embodiments of the present disclosure, Zadno-Aziz may prefer to leave the therapy device in place even if the injected substance is to be removed. In many cases, there may be no need to remove any antiproliferative agent used with the embodiments of the present disclosure, again a distinction from the method of Zadno-Azizi. The fluid containing the embolic material must be withdrawn for the Zadno-Azizi to be effective less the embolic material embolizes downstream. The success of the embodiments of the present disclosure may not be predicated on removal of any injected drug, as the drug may be released downstream where it likely would be harmless to the tissues.

Even more important in differentiating the embodiments of the present disclosure from the method of Zadno-Azizi may be the timing aspect. The fluid injected and aspirated is done after the therapeutic intervention with the Zadno-Azizi method whereas in the present methods, an agent is used to inhibit restenosis. The agent used may be, but not limited to paclitaxel, and the agent is injected before the therapeutic procedure and left in place during the therapeutic procedure. The antiproliferative agent may or may not be aspirated subsequent to the therapeutic procedure.

Moreover, the prior art devices of Ya and Zadno-Azizi both use a distal occluder with a hollow lumen, which is needed to inflate the distal balloon. Embodiments of the present disclosure may have no need for this feature when the distal occluder may be a mechanical blocking element so that they may be no need for a hollow lumen along the distal occluder.

The balloon stent assembly according to many embodiments, in contrast with known the temporary stents, will both dilate the plaque in a controlled manner using the balloon, which causes little injury to the vessel, and supports the vessel for an extended period of time using the temporary stent. Known temporary stents are commonly intended to only support the vessel after something untoward happens during the procedure, i.e., dissection, vasospasm, or vasoconstriction. Embodiments of the present disclosure, because all of the functions (dilatation and support functions) happen more or less simultaneously, prevents noticeable dissections, vasospasm, or vasoconstriction as the vessel wall is supported during and immediately after the intervention, a great improvement over the prior art device. There may be virtually no time for the untoward events to occur with the current devices as there is no time that the vessel wall does not have radial force being exerted upon it. Moreover, embodiments of the present disclosure may prevent acute elastic recoil which may be due to many other factors other than dissection, vasospasm, or vasoconstriction.

The above descriptions may have used terms above, below, top, bottom, over, under, et cetera. These terms may be used in the description and claims to aid understanding of the inventions of the present disclosure and not used in a limiting sense.

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed in practicing the inventions of the present disclosure. It is intended that the following claims define the scope of the invention and that methods and structures within the scope of these claims and their equivalents be covered thereby.

1. A method of treating a target region within a bodily lumen, the method comprising:
advancing a stent-like tubular scaffold assembly over an expandable element on a distal portion of a catheter;
affixing the stent-like tubular scaffold assembly to an elongate shaft of the catheter;
positioning the catheter and the stent-like tubular scaffold assembly at or near the target region;
expanding the expandable element of the catheter to press the expandable element and the stent-like tubular scaffold assembly against the target site;
collapsing the expandable element while leaving the stent-like tubular scaffold assembly expanded against the target site, wherein the expanded stent-like tubular scaffold assembly maintains sufficient pressure against an inner wall of the bodily lumen at the target site to inhibit elastic recoil thereof; and
collapsing the stent-like tubular scaffold assembly.

2.-21. (canceled)

22. A stent-like tubular scaffold assembly for treating a target region with a bodily lumen, the assembly comprising:
a stent-like tubular scaffold configured to be advancable over an elongate shaft of a catheter to be positioned over an expandable member of the catheter, the stent-like tubular scaffold having a proximal end and a distal end; and
one or more of a proximal affixation member at the proximal end or a distal affixation member at the distal end, wherein the proximal affixation member or the distal affixation member is configured to couple to the elongate shaft of the catheter.

23.-32. (canceled)
